(19) United States
(12) Patent Application Publication
(10) Pub. No.: US 2010/0247590 A1
(43) Pub. Date: Sep. 30, 2010

(54) Peptide-Based Systems For Delivery Of Cosmetic Agents

(75) Inventors: Douglas Robert Anton, Wilmington, DE (US); Susan Daly, Basking Ridge, NJ (US); Robert J. Bianchini, Hillsborough, NJ (US); Hong Wang, Kennett Square, PA (US); Pierre E. Rouviere, Wilmington, DE (US); Scott D. Cunningham, Chadds Ford, PA (US); Stephen R. Fahnestock, Wilmington, DE (US); Tanja Maria Gruber, Media, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignees: JOHNSON & JOHNSON, New Brunswick, NJ (US); E. I. DU PONT DE NEMOURS AND COMPANY, Wilmington, DE (US)

(21) Appl. No.: 12/748,685

(22) Filed: Mar. 29, 2010

Related U.S. Application Data
(60) Provisional application No. 61/164,533, filed on Mar. 30, 2009.

(51) Int. Cl. A61K 8/02; A61K 9/14; A61Q 17/04; A61K 8/64; A61P 17/10; A61P 31/10; A61P 31/04; A61Q 9/00; A61Q 5/00

(52) U.S. Cl. 424/401; 424/489; 424/59; 424/70.9; 424/70.6; 512/4

(57) ABSTRACT

The present invention relates to compositions and systems comprising peptide-based reagents for delivery of cosmetic benefit agents to human hair, human skin, or human nail.

### CROSS REFERENCE TO RELATED APPLICATIONS

This application claims the benefit of U.S. Provisional Application No. 61/164,533, filed Mar. 30, 2009, the entirety of which is incorporated herein.

### TECHNICAL FIELD

The present invention relates to compositions and systems comprising peptide-based reagents for delivery of cosmetic benefit agents to human hair, human skin, and/or human nail.

### BACKGROUND

Many cosmetic care products are comprised of one or more particulate benefit agents, for example, coloring agents and conditioning agents that improve the cosmetic properties of keratin-containing body surfaces such as hair, skin, and nails. These particulate benefit agents do not durably bind to these body surfaces. As a result, these products need to be frequently reapplied to the body surface in order to maintain the desired effect.

Various protein-based reagents have been developed in attempts to improve the binding durability of the benefit agent for a target surface or for targeted delivery of a benefit agent to a target surface. Overall, these are impractical and many of these “binding” proteins are expensive and difficult to prepare and include, for example, immunoglobulins, immunoglobulin-derived proteins, and non-immunoglobulin binding proteins that require a complex support scaffold for effective binding (See Binz, H. et al. (2005) Nature Biotechnology 23, 1257-1268 for a review of various scaffold-assisted approaches).

Single chain peptides and peptide-based reagents have been reported for use in cosmetic applications, for example, traditional colorants and conditioners for hair, skin, and nails (Huang et al. in U.S. Pat. No. 7,220,405 and U.S. Patent Application Publication Nos. US2005/0226839, US2007/0053837, and US2008/0152600; Wang et al. in U.S. Patent Application Publication Nos. US2007/0196395 and US2006/0199206; O'Brien et al. in U.S. Patent Application Publication No. US2006/0073111, U.S. Pat. No. 7,285,264 and Published PCT Application No. WO2008/054746; Beck et al. in U.S. Patent Application Publication No. US2007/0065387; Fahnestock et al. in U.S. Patent Application Publication No. US2008/0107614; and Benson et al. in U.S. patent application Ser. Nos. 12/198,358 and 12/198,382), carbon nanotube-based hair colorants (Huang et al. in U.S. Patent Application Publication No. US2005/0229335), skin sunscreen agents (Buseman-Williams et al. in U.S. Pat. No. 7,309,482 and Lowe et al. in U.S. Patent Application Publication No. US2007/0110686), hair sunscreen agents (Beck et al. in U.S. Patent Application Publication No. 2008/0175798), antidandruff agents (O'Brien et al in U.S. patent application Ser. No. 12/273,753), and antiacne agents (O'Brien et al. in U.S. patent application Ser. No. 12/273,778).

Certain single chain binding peptides with strong affinity for hair, skin, and/or nails have been identified using phage display (Huang et al., supra; Estell et al. in Published PCT Application No. WO01/79479; Murray et al. in U.S. Patent Application Publication No. 2002/0098524; Janssen et al., U.S. Patent Application Publication No. 2003/0152976; and Janssen et al. in Published PCT Application No. WO04/048399). Additionally, empirically-generated hair and skin-binding peptides that are based on positively charged amino acids have been reported (WO 2004/000257 to Rothe et al.). These approaches illustrate that short, linear peptides lacking complex scaffolds and/or immunoglobulin-like structures can have strong affinity (i.e., K_{d}<10⁻⁵ M) for the surface of hair, skin, and/or nails.

The binding strength, however, of a single, short peptide may not be sufficient to meet the durability required for most cosmetic applications. In those applications, two or more of the identified surface-binding peptides can be linked together to prepare linear binding domains (also referred to herein as “hands”) having an increased affinity for the targeted surface (i.e., skin, hair, or nails). Often, two or more binding domains may be linked via short peptide spacers separating the individual target surface-binding peptides.

Peptides having multiple, rationally-designed binding domains have been reported wherein each domain was designed to couple together at least two substrates, wherein at least one of the binding domains was designed to have affinity for at least one keratin-containing body substrate (e.g., hair, skin, and/or nails) while the second binding domain was designed to have affinity for a benefit agent (U.S. Published Patent Application No. US2007/0065387 to Beck et al. and U.S. Pat. No. 7,285,264 to O'Brien et al.; and U.S. Patent Application Publication No. 2003/0185870 to Grinstaff et al.). These peptide-based reagents comprise at least two binding domains (referred to herein as “two-handed peptides” or “two-handed peptide-based reagents”), wherein each binding domain has been designed to have an affinity for their respective substrate

There remains a need for selective linear peptides and cosmetic systems that can effectively couple a benefit agent to human hair, skin, and/or nails and that are not “two-handed.” There also remains a need for selective peptides wherein the benefit agent can be unbound from the peptide using mild conditions.

### SUMMARY

The present invention is directed to cosmetic systems comprising a peptidic component comprising at least one binding domain which binds to at least one of human hair, human skin or human nail with a K_{d} or MB₅₀ value of 10⁻⁵ molar or less and which further comprises the first part of an affinity pair; and a stable dispersion of particulate benefit agent having average particle size of between about 0.01 micron and about 75 microns and the second part of the affinity pair; the at least one binding domain has a greater binding affinity for the human hair, skin or nail than it has for the particles of the dispersion. Methods of using the cosmetic systems of the invention are also described, including the application of a benefit agent and the removal of the benefit agent from at least one of human hair, skin or nail.

### BRIEF DESCRIPTION OF THE DRAWINGS

FIG. 1 depicts the fluorescence of one embodiment of the invention, biotinylated HCP5 dimer peptide, bound the particles to hair.

FIG. 2 is a histogram demonstrating hair coloring ability of certain embodiments of the invention using silica-coated iron oxide pigment.

FIG. 3A depicts hair tresses treated with according to the procedures set forth in Example 7 after initial exposure to coated iron oxide particles.

FIG. 3B depicts hair tresses treated according to the procedures set forth in Example 7, followed by washing with water.

FIG. 3C depicts hair tresses treated according to the procedures set forth in Example 7, followed by washing with 0.25% SLES Wash.

FIG. 4A is an electron micrograph of a hair treated with (HCP5)2-biotin and 500 nm streptavidin-coated iron oxide particles.

FIG. 4B is an electron micrograph of a hair treated with (HCP5)2-biotin and 200 nm streptavidin-coated iron oxide particle.

### SEQUENCE DESCRIPTIONS

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5 (a-bis), and Section 208 and Annex C of the Administrative Instructions). The symbols and format used for nucleotide and amino acid sequence data comply with the rules set forth in 37 C.F.R. §1.822.

SEQ ID NOs: 1-184 and 186-189 are amino acid sequences of keratin-containing body surface-binding peptides. SEQ ID NOs: 1-134 and 184 are amino acid sequences of hair-binding peptides. SEQ ID NOs: 130-134 are empirically-generated sequences that bind to hair and skin. SEQ ID NOs: 135-182 are amino acid sequences of skin-binding peptides. SEQ ID NOs: 183-184 are amino acid sequences to nail-binding peptides.

SEQ ID NO: 185 is the amino acid sequence of peptide (“HAT”) tag that binds to metal ions.

SEQ ID NO: 186 is the amino acid sequence of SEQ ID NO: 76 with a C-terminal lysine residue.

SEQ ID NO: 187 is the amino acid sequence of SEQ ID NO: 82 with a C-terminal lysine residue.

SEQ ID NO: 188 is the amino acid sequence of SEQ ID NO: 86 with a C-terminal lysine residue.

SEQ ID NO: 189 is the amino acid sequence of SEQ ID NO: 110 with a C-terminal lysine residue.

SEQ ID NO: 190 is the amino acid sequence of a peptide comprising a randomized version of SEQ ID NO: 82.

SEQ ID NO: 191 is the amino acid sequence of a peptide comprising a randomized version of SEQ ID NO: 120.

SEQ ID NO: 192 is the amino acid sequence of a peptide comprising a randomized version of SEQ ID NO: 30.

SEQ ID NO: 193 is the amino acid sequence of a pigment binding peptide.

SEQ ID NO: 194 is the amino acid sequence of a cellulose acetate binding peptide.

SEQ ID NO: 195 is the amino acid sequence of a cellulose acetate binding peptide.

SEQ ID NO: 196 is the amino acid sequence of peptide HC263.

SEQ ID NO: 197 is the amino acid sequence of peptide HC264.

SEQ ID NO: 198 is the amino acid sequence of peptide HC214.

SEQ ID NO: 199 is the amino acid sequence of peptide HC204.

SEQ ID NO: 200 is the amino acid sequence of peptide HC205.

SEQ ID NO: 201 is the amino acid sequence of peptide HC352.

SEQ ID NO: 202 is the amino acid sequence of peptide HC423.

SEQ ID NO: 203 is the amino acid sequence of peptide HC424.

SEQ ID NO: 204 is the amino acid sequence of peptide (HCP5)2 also referred to herein as “HCP5-dimer”.

SEQ ID NO: 205 is the amino acid sequence of peptide of SEQ ID NO: 204 with a C-terminal lysine.

SEQ ID NO: 206 is the amino acid sequence of a streptavidin-binding peptide tag.

SEQ ID NO: 207 is the amino acid sequence of peptide HC260.

SEQ ID NO: 208 is the amino acid sequence of the peptide linker “tonB”.

SEQ ID NO: 209 is the amino acid sequence of a peptide bridge.

SEQ ID NO: 210 is the amino acid sequence of peptide HC353.

SEQ ID NO: 211 is the amino acid sequence of peptide HC634.

SEQ ID NO: 212 is the amino acid sequence of peptide HC635.

SEQ ID NO: 213 is the amino acid sequence of peptide HC636.

SEQ ID NO: 214 is the amino acid sequence of peptide HC637.

SEQ ID NO: 215 is the amino acid sequence of peptide HC638.

SEQ ID NO: 216 is the amino acid sequence of peptide HC639.

SEQ ID NO: 217 is the amino acid sequence of peptide HC640.

SEQ ID NO: 218 is the amino acid sequence of peptide HC641.

SEQ ID NO: 219 is the amino acid sequence of peptide HC642.

SEQ ID NO: 220 is the amino acid sequence of peptide HC643.

SEQ ID NO: 221 is the amino acid sequence of peptide HC644.

SEQ ID NO: 222 is the amino acid sequence of peptide HC645.

SEQ ID NOs: 223-234 are amino acid sequences of peptides designed to electrostatically associate with a pigment surface.

SEQ ID No: 235 is the amino acid sequence of peptide HHHHHH.

SEQ ID NO: 225 is the amino acid sequence of SEQ ID NO: 212 with a C-terminal polylysine block.

### DETAILED DESCRIPTION OF ILLUSTRATIVE EMBODIMENTS

Improved cosmetic systems for applying a benefit agent to at least one of human hair, human skin, or human nail have been developed and are described herein. Methods of preparing and using these systems are also described. The cosmetic systems of the present invention comprise a peptidic component and a particulate benefit agent provided in a stable particulate dispersion.

### The Peptidic Component

The peptidic component comprises at least one peptidic binding domain that binds to at least one human body surface that is human hair, human skin, or human nail. Certain peptidic components of the invention will be about 500 amino acids, or less, in length. The peptidic component can be provided as, for example, an aqueous solution, a powder, an emulsion, a suspension, a dispersion, a gel, a cream, or an aerosol. The peptidic component may be applied to at least one of human hair, skin, or nail at a concentration of about 0.01% to about 10%, in some embodiments, about 0.01% to about 5%, by weight of the total composition.

Within the scope of the invention, the terms “peptide,” “peptidic,” and “polypeptide” will be used interchangeably to refer to a polymer of two or more amino acids joined together by a peptide bond, wherein the peptide is of unspecified length. Peptides, oligopeptides, and polypeptides are included within the present definition. In one aspect, this term also includes post expression modifications of the peptide, for example, glycosylations, acetylations, phosphorylations, and the like. Also included within the definition are, for example, peptides containing one or more analogues of an amino acid or labeled amino acids and peptidomimetics. In exemplary embodiments, the peptidic component will comprise 15 to 500, 15 to 250, or 15 to 100 amino acids. The following abbreviations will be used to identify specific amino acids.

| | 3-Letter | 1-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid | Xaa | X |
| (or as defined herein) | | |

As used herein, “binding domain” refers to a peptide comprising one, ideally two or more shorter peptides (“subdomains”) that have been identified as having an affinity for a particular surface or surfaces, for example, human hair, skin, and/or nails. In certain embodiments, a binding domain may include from 2 to about 50 or 2 to about 25, of the shorter peptides. Other embodiments include those having binding domains including 2 to about 10 shorter peptides. Other embodiments are those binding domains including 2, 3, 4, or 5 shorter peptides.

These shorter peptides may be directly linked to each other to form a binding domain or may be linked via one or more short peptide spacers to form a binding domain. In some embodiments, peptide spacers are from 1 to 100 or 1 to 50, amino acids in length. In other embodiments, the peptide spacers are about 1 to about 25, 3 to about 40, or 3 to about 30 amino acids in length. In still other embodiments are spacers that are about 5 to about 20 amino acids in length.

In certain embodiments, the binding domain of the invention binds to at least one of human hair, skin, and nail with a binding affinity value of 10⁻⁵ molar (M) or less. In some embodiments, the peptidic binding domains will have a binding affinity value of 10⁻⁵ or less in the presence of at least about 50-500 mM salt. The term “binding affinity” refers to the strength of the interaction of a binding peptide with its respective substrate, in this case, human hair, skin, or nail. Binding affinity can be defined or measured in terms of the binding peptide's dissociation constant (“K_{d}”), or “MB₅₀.”

“K_{d}” corresponds to the concentration of peptide at which the binding site on the target is half occupied, i.e., when the concentration of target with peptide bound (bound target material) equals the concentration of target with no peptide bound. The smaller the dissociation constant, the more tightly the peptide is bound. For example, a peptide with a nanomolar (nM) dissociation constant binds more tightly than a peptide with a micromolar (μM) dissociation constant. Certain embodiments of the invention will have a K_{d} value of 10⁻⁵ or less.

“MB₅₀” refers to the concentration of the binding peptide that gives a signal that is 50% of the maximum signal obtained in an ELISA-based binding assay. See, e.g., Example 3 of U.S. Patent Application Publication 2005/022683; hereby incorporated by reference. The MB₅₀ provides an indication of the strength of the binding interaction or affinity of the components of the complex. The lower the value of MB₅₀, the stronger, i.e., “better,” the interaction of the peptide with its corresponding substrate. For example, a peptide with a nanomolar (nM) MBₛₒ binds more tightly than a peptide with a micromolar (μM) MB₅₀. Certain embodiments of the invention will have a MB₅₀ value of 10⁻⁵ or less.

In some embodiments, the peptidic binding domains have a binding affinity, as measured by K_{d} or MB₅₀ values, of less than or equal to about 10⁻⁵ M, less than or equal to about 10⁻⁶ M, less than or equal to about 10⁻⁷ M, less than or equal to about 10⁻⁸ M, less than or equal to about 10⁻⁹ M, or less than or equal to about 10⁻¹⁰ M. Peptides that have been identified to bind to at least human hair are also referred to as “hair-binding peptides (HBP).” Peptides that have been identified to bind to at least human skin are also referred to as “skin-binding peptides (SBP).” Peptides that have been identified to bind to at least human nail are also referred to as “nail-binding peptides (NBP).”

In some embodiments, peptidic binding domains are comprised of peptidic binding subdomains that are up to about 60 amino acids in length. Certain embodiment will have peptidic bind subdomains that are 7 to about 60 amino acids in length. In other embodiments are those peptidic binding subdomains that are 7 to 50 or 7 to 30 amino acids in length. In still other embodiments are those peptidic binding subdomains that are 7 to 27 amino acids in length.

While peptidic components comprising a single hair-, skin-, and/or nail-binding domain are certain embodiments of the invention, in other embodiments of the invention, it may be advantageous that the peptidic component comprise more than one binding domain that binds to at least one of human hair, human skin, or human nail. The inclusion of multiple, i.e., two or more, binding domains can provide a peptidic component that is, for example, even more cosmetically durable than those peptidic components including a single binding domain. In some embodiments, the peptidic component includes from 2 to about 50 or 2 to about 25 peptidic binding domains. Other embodiments include those peptidic components including 2 to about 10 or 2 to 5 peptidic binding domains.

The multiple binding domains can be linked directly together or they can be linked together using peptide spacers. Certain peptide spacers are from 1 to 100 or 1 to 50 amino acids in length. In some embodiments, the peptide spacers are about 1 to about 25, 3 to about 40, or 3 to about 30 amino acids in length. In other embodiments are spacers that are about 5 to about 20 amino acids in length.

The binding domains of the invention will also have a greater binding affinity for the human hair, skin, or nail than they have for the particles of the dispersion. This preferential affinity of the binding domains for the human hair, skin, or nail over the particles of the dispersion results in a cosmetic system wherein a greater percentage of the binding domains are available for binding to the at least one of human hair skin or nail as compared to those systems wherein the binding domains do not have a greater binding affinity for the human hair, skin or nail over the particles of the dispersion. In some embodiments, the binding domains have at least about a 2-fold greater (i.e., about 2 times greater) binding affinity for the human hair, skin, or nail than they have for the particles of the dispersion. In other embodiments, the binding domains have at least a 5-fold greater (i.e., about 5-times greater) binding affinity for the human hair, skin, or nail than they have for the particles of the dispersion. In still other embodiments, the binding domains have at least a 10-fold greater (i.e., about 10-times greater) binding affinity for the human hair, skin, or nail than they have for the particles of the dispersion.

Peptidic binding domains, and the shorter peptides of which they are comprised, can be identified using any number of methods known to those skilled in the art, including, for example, any known biopanning techniques such as phage display, bacterial display, yeast display, ribosome display, mRNA display, and combinations thereof.

The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500); ribosome display (U.S. Pat. No. 5,643,768; U.S. Pat. No. 5,658,754; and U.S. Pat. No. 7,074,557), and mRNA display technology (PROFUSION™; U.S. Pat. No. 6,258,558; U.S. Pat. No. 6,518,018; U.S. Pat. No. 6,281,344; U.S. Pat. No. 6,214,553; U.S. Pat. No. 6,261,804; U.S. Pat. No. 6,207,446; U.S. Pat. No. 6,846,655; U.S. Pat. No. 6,312,927; U.S. Pat. No. 6,602,685; U.S. Pat. No. 6,416,950; U.S. Pat. No. 6,429,300; U.S. Pat. No. 7,078,197; and U.S. Pat. No. 6,436,665). Techniques to generate such biological peptide libraries are described in Dani, M., J. of Receptor & Signal Transduction Res., 21(4):447-468 (2001).

One method to randomly generate peptides is by phage display. Since its introduction in 1985, phage display has been widely used to discover a variety of ligands including peptides, proteins and small molecules for drug targets (Dixit, J. of Sci. & Ind. Research, 57:173-183 (1998)). The applications have expanded to other areas such as studying protein folding, novel catalytic activities, DNA-binding proteins with novel specificities, and novel peptide-based biomaterial scaffolds for tissue engineering (Hoess, Chem. Rev. 101:3205-3218 (2001) and Holmes, Trends Biotechnol. 20:16-21 (2002)). Whaley et al. (Nature 405:665-668 (2000)) discloses the use of phage display screening to identify peptide sequences that can bind specifically to different crystallographic forms of inorganic semiconductor substrates.

A modified screening method that comprises contacting a peptide library with an anti-target to remove peptides that bind to the anti-target, then contacting the non-binding peptides with the target has been described (Estell et al. WO 01/079479, Murray et al. U.S. Patent Application Publication No. 2002/0098524, and Janssen et al. U.S. Patent Application Publication No. 2003/0152976). Using the target/anti-target method, a peptide sequence that preferentially binds to hair and not to skin and a peptide sequence that preferentially binds to skin and not hair can be identified. Using the same method, Janssen et al. (WO 04/048399) identified other keratin-contain body surface-binding peptides (i.e., skin-binding and hair-binding peptides), as well as several other binding motifs.

Phage display is a selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. As used herein, “biopanning” may be used to describe any selection procedure (phage display, ribosome display, mRNA-display, etc.) where a library of displayed peptides a library of displayed peptides is panned against a specified target material (e.g. hair). In its simplest form, phage display biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

The peptidic binding domains can also be empirically generated from sequences reported to have affinity for at least one of human hair, skin, or nail. For example, peptides having an affinity for a human body surface have been described in U.S. Pat. Nos. 7,220,405 and 7,285,264; U.S. Patent Application Publications Nos. US 2005-0226839, US 2005-0249682, US 2007-0065387, US 2007-0067924, US 2007-0196305, US 2007-0110686, US 2006-0073111, and US 2006-0199206; U.S. patent application Ser. No. 11/877,692; U.S. Patent Application Publication No. 2008-0175798; and PCT Patent Application Publication No. WO2004048399. Human body surface peptides are also set forth in Tables 1A-1F.

TABLE 1A

| Body | | | |
|---|---|---|---|
| Surface | Amino Acid Sequence | SEQ ID NO: | Reference |
| Hair | RVPNKTVTVDGA | 1 | US 2005-0226839 |
| | | | U.S. Pat. No. 7,220,405 |
| Hair | DRHKSKYSSTKS | 2 | US 2005-0226839 |
| | | | U.S. Pat. No. 7,220,405 |
| Hair | KNFPQQKEFPLS | 3 | US 2005-0226839 |
| | | | U.S. Pat. No. 7,220,405 |
| Hair | QRNSPPAMSRRD | 4 | US 2005-0226839 |
| | | | U.S. Pat. No. 7,220,405 |
| Hair | TRKPNMPHGQYL | 5 | US 2005-0226839 |
| | | | U.S. Pat. No. 7,220,405 |
| Hair | KPPHLAKLPFTT | 6 | US 2005-0226839 |
| | | | U.S. Pat. No. 7,220,405 |
| Hair | NKRPPTSHRIHA | 7 | US 2005-0226839 |
| | | | U.S. Pat. No. 7,220,405 |
| Hair | NLPRYQPPCKPL | 8 | US 2005-0226839 |
| | | | U.S. Pat. No. 7,220,405 |
| Hair | RPPWKKPIPPSE | 9 | US 2005-0226839 |
| | | | U.S. Pat. No. 7,220,405 |
| Hair | RQRPKDHFFSRP | 10 | US 2005-0226839 |
| | | | U.S. Pat. No. 7,220,405 |
| Hair | SVPNKXVTVDGX | 11 | US 2005-0226839 |
| | | | U.S. Pat. No. 7,220,405 |
| Hair | TTKWRHRAPVSP | 12 | US 2005-0226839 |
| | | | U.S. Pat. No. 7,220,405 |
| Hair | WLGKNRIKPRAS | 13 | US 2005-0226839 |
| | | | U.S. Pat. No. 7,220,405 |
| Hair | SNFKTPLPLTQS | 14 | US 2005-0226839 |
| | | | U.S. Pat. No. 7,220,405 |
| Hair | SVSVGMKPSPRP | 15 | US 2005-0226839 |
| | | | U.S. Pat. No. 7,220,405 |
| Hair | DLHTVYH | 16 | US 2005-0226839 |
| | | | U.S. Pat. No. 7,220,405 |
| Hair | HIKPPTR | 17 | US 2005-0226839 |
| | | | U.S. Pat. No. 7,220,405 |
| Hair | HPVWPAI | 18 | US 2005-0226839 |
| | | | U.S. Pat. No. 7,220,405 |
| Hair | MPLYYLQ | 19 | US 2005-0226839 |
| | | | U.S. Pat. No. 7,220,405 |
| Hair | HLTVPWRGGGSAVPFYSHSQI | 20 | US 2005-0226839 |
| | TLPNH | | U.S. Pat. No. 7,220,405 |
| Hair | GPHDTSSGGVRPNLHHTSKKE | 21 | US 2005-0226839 |
| | KRENRKVPFYSHSVTSRGNV | | U.S. Pat. No. 7,220,405 |
| Hair | KHPTYRQ | 22 | US 2005-0226839 |
| | | | U.S. Pat. No. 7,220,405 |
| Hair | HPMSAPR | 23 | US 2005-0226839 |
| | | | U.S. Pat. No. 7,220,405 |
| Hair | MPKYYLQ | 24 | US 2005-0226839 |
| | | | U.S. Pat. No. 7,220,405 |
| Hair | MHAHSIA | 25 | US 2005-0226839 |
| | | | U.S. Pat. No. 7,220,405 |
| Hair | AKPISQHLQRGS | 26 | US 2005-0226839 |
| | | | U.S. Pat. No. 7,220,405 |
| Hair | APPTPAAASATT | 27 | US 2005-0226839 |
| | | | U.S. Pat. No. 7,220,405 |
| Hair | DPTEGARRTIMT | 28 | US 2005-0226839 |
| | | | U.S. Pat. No. 7,220,405 |
| Hair | LDTSFPPVPFHA | 29 | US 2005-0226839 |
| | | | U.S. Pat. No. 7,220,405 |
| Hair | LDTSFHQVPFHQ | 30 | US 2005-0226839 |
| | | | U.S. Pat. No. 7,220,405 |
| Hair | LPRIANTWSPS | 31 | US 2005-0226839 |
| | | | U.S. Pat. No. 7,220,405 |
| Hair | RTNAADHPAAVT | 32 | US 2005-0226839 |
| | | | U.S. Pat. No. 7,220,405 |
| | | | US 2007-0065387 |
| Hair | SLNWVTIPGPKI | 33 | US 2005-0226839 |
| | | | U.S. Pat. No. 7,220,405 |
| Hair | TDMQAPTKSYSN | 34 | US 2005-0226839 |
| | | | U.S. Pat. No. 7,220,405 |
| Hair | TIMTKSPSLSCG | 35 | US 2005-0226839 |
| | | | U.S. Pat. No. 7,220,405 |
| Hair | TPALDGLRQPLR | 36 | US 2005-0226839 |
| | | | U.S. Pat. No. 7,220,405 |
| Hair | TYPASRLPLLAP | 37 | US 2005-0226839 |
| | | | U.S. Pat. No. 7,220,405 |
| Hair | AKTHKHPAPSYS | 38 | US 2005-0226839 |
| | | | U.S. Pat. No. 7,220,405 |
| Hair | TDPTPFSISPER | 39 | US 2005-0226839 |
| | | | U.S. Pat. No. 7,220,405 |
| Hair | SQNWQDSTSYSN | 40 | US 2005-0226839 |
| | | | U.S. Pat. No. 7,220,405 |
| Hair | WHDKPQNSSKST | 41 | US 2005-0226839 |
| | | | U.S. Pat. No. 7,220,405 |
| Hair | LDVESYKGTSMP | 42 | US 2005-0226839 |
| | | | U.S. Pat. No. 7,220,405 |
| Hair | NTPKENW | 43 | WO2004048399 |
| Hair | NTPASNR | 44 | WO2004048399 |
| Hair | PRGMLST | 45 | WO2004048399 |
| Hair | PPTYLST | 46 | WO2004048399 |
| Hair | TIPTHRQHDYRS | 47 | WO2004048399 |
| Hair | TPPTHRL | 48 | WO2004048399 |
| Hair | LPTMSTP | 49 | WO2004048399 |
| Hair | LGTNSTP | 50 | WO2004048399 |
| Hair | TPLTGSTNLLSS | 51 | WO2004048399 |
| Hair | TPLTKET | 52 | WO2004048399 |
| Hair | KQSHNPP | 53 | WO2004048399 |
| Hair | QQSHNPP | 54 | WO2004048399 |
| Hair | TQPHNPP | 55 | WO2004048399 |
| Hair | STNLLRTSTVHP | 56 | WO2004048399 |
| Hair | HTQPSYSSTNLF | 57 | WO2004048399 |
| Hair | SLLSSHA | 58 | WO2004048399 |
| Hair | QQSSISLSSHAV | 59 | WO2004048399 |
| Hair | NASPSSL | 60 | WO2004048399 |
| Hair | HSPSSLR | 61 | WO2004048399 |
| Hair | K H/R/N SHHTH | 62 | WO2004048399 |
| Hair | E H/R/N SHHTH | 63 | WO2004048399 |
| Hair | SHHTHYGQPGPV | 64 | WO2004048399 |
| Hair | LESTSLL | 65 | WO2004048399 |
| Hair | DLTLPFH | 66 | US 2007-0065387 |
| Hair | RTNAADHP | 67 | US 2007-0067924 |
| Hair | IPWWNIRAPLNA | 68 | US 2007-0067924 |
| Hair | EQISGSLVAAPWEGEGER | 69 | U.S. Pat. No. 11/877,692 |
| Hair | PKRGRHKRPKRHKGGGTPPEL | 70 | U.S. Pat. No. 11/877,692 |
| | LHGAPRSC | | |
| Hair | LDTSFHQVPFHQKRKRKD | 71 | U.S. Pat. No. 11/877,692 |
| Hair | EQISGSLVAAPWKRKRKD | 72 | U.S. Pat. No. 11/877,692 |
| Hair | TPPELLHGDPRSKRKRKD | 73 | U.S. Pat. No. 11/877,692 |
| Hair | NTSQLSTEGEGED | 74 | U.S. Pat. No. 11/877,692 |
| Hair | TPPELLHGDPRSC | 75 | US 2007-0067924 |
| Hair | HINKTNPHQGNHHSEKTQRQ | 76 | US 2008-0175798 |
| Hair | HAHKNQKETHQRHAA | 77 | US 2008-0175798 |
| Hair | HEHKNQKETHQRHAA | 78 | US 2008-0175798 |
| | | | U.S. Pat. No. 7,285,264 |
| Hair | HNHMQERYTEPQHSPSVNGL | 79 | US 2008-0175798 |
| Hair | THSTHNHGSPRHTNADA | 80 | US 2007-0196305 |
| Hair | GSCVDTHKADSCVANNGPAT | 81 | US 2008-0175798 |
| Hair | AQSQLPDKHSGLHERAPQRY | 82 | US 2008-0175798 |
| Hair | AQSQLPAKHSGLHERAPQRY | 83 | US 2008-0175798 |
| Hair | AQSQLPEKHSGLHERAPQRY | 84 | US 2008-0175798 |
| Hair | TDMMHNHSDNSPPHRRSPRN | 85 | US 2008-0175798 |
| Hair | TPPELAHTPHHLAQTRLTDR | 86 | US 2008-0175798 |
| Hair | RLLRLLRLLRLL | 87 | US 2008-0175798 |
| Hair | TPPELLHGEPRS | 88 | US 2008-0175798 |
| Hair | TPPELLHGAPRS | 89 | U.S. Pat. No. 7,285,264 |
| Hair | EQISGSLVAAPW | 90 | US 2005-0226839 |
| (normal and | | | U.S. Pat. No. 7,220,405 |
| bleached) | | | |
| Hair | NEVPARNAPWLV | 91 | US 2005-0226839 |
| (bleached) | | | U.S. Pat. No. 7,220,405 |
| Hair | NSPGYQADSVAIG | 92 | US 2005-0226839 |
| (bleached) | | | U.S. Pat. No. 7,220,405 |
| Hair | AKPISQHLQRGS | 93 | US 2005-0226839 |
| (bleached) | | | U.S. Pat. No. 7,220,405 |
| Hair | LDTSFPPVPFHA | 94 | US 2005-0226839 |
| (bleached) | | | U.S. Pat. No. 7,220,405 |
| Hair | SLNWVTIPGPKI | 95 | US 2005-0226839 |
| (bleached) | | | U.S. Pat. No. 7,220,405 |
| Hair | TQDSAQKSPSPL | 96 | US 2005-0226839 |
| (bleached) | | | U.S. Pat. No. 7,220,405 |
| Hair | KELQTRNVVQRE | 97 | US 2005-0226839 |
| (bleached) | | | U.S. Pat. No. 7,220,405 |
| Hair | QRNSPPAMSRRD | 98 | US 2005-0226839 |
| (bleached) | | | U.S. Pat. No. 7,220,405 |
| Hair | TPTANQFTQSVP | 99 | US 2005-0226839 |
| (bleached) | | | U.S. Pat. No. 7,220,405 |
| Hair | AAGLSQKHERNR | 100 | US 2005-0226839 |
| (bleached) | | | U.S. Pat. No. 7,220,405 |
| Hair | ETVHQTPLSDRP | 101 | US 2005-0226839 |
| (bleached) | | | U.S. Pat. No. 7,220,405 |
| Hair | KNFPQQKEFPLS | 102 | US 2005-0226839 |
| (bleached) | | | U.S. Pat. No. 7,220,405 |
| Hair | LPALHIQRHPRM | 103 | US 2005-0226839 |
| (bleached) | | | U.S. Pat. No. 7,220,405 |
| Hair | QPSHSQSHNLRS | 104 | US 2005-0226839 |
| (bleached) | | | U.S. Pat. No. 7,220,405 |
| Hair | RGSQKSKPPRPP | 105 | US 2005-0226839 |
| (bleached) | | | U.S. Pat. No. 7,220,405 |
| Hair | THTQKTPLLYYH | 106 | US 2005-0226839 |
| (bleached) | | | U.S. Pat. No. 7,220,405 |
| Hair | TKGSSQAILKST | 107 | US 2005-0226839 |
| (bleached) | | | U.S. Pat. No. 7,220,405 |
| Hair | TAATTSP | 108 | US 2005-0226839 |
| (normal and | | | U.S. Pat. No. 7,220,405 |
| bleached) | | | |
| Hair | LGIPQNL | 109 | US 2005-0226839 |
| (bleached) | | | U.S. Pat. No. 7,220,405 |
| Hair | THSTHNHGSPRHTNADAGNP | 110 | US 2007-0065387 |
| (Conditioner | | | US 2007-0196305 |
| resistant) | | | |
| Hair | QQHKVHHQNPDRSTQDAHHS | 111 | US 2007-0196305 |
| (Conditioner | | | |
| resistant) | | | |
| Hair | HHGTHHNATKQKNHV | 112 | US 2007-0196305 |
| (Conditioner | | | |
| resistant) | | | |
| Hair | STLHKYKSQDPTPHH | 113 | US 2007-0196305 |
| (Conditioner | | | |
| resistant) | | | |
| Hair | SVSVGMKPSPRP | 114 | US 2007-0196305 |
| (Conditioner | | | |
| resistant) | | | |
| Hair | TPPTNVLMLATK | 115 | US 2006-0073111 |
| (shampoo | | | |
| resistant) | | | |
| Hair | TPPELLHGDPRS | 116 | US 2006-0073111 |
| (shampoo | | | |
| resistant) | | | |
| Hair | NTSQLST | 117 | US 2007-0067924 |
| (shampoo | | | U.S. Pat. No. 7,285,264 |
| resistant) | | | |
| Hair | STLHKYKSQDPTPHH | 118 | US 2007-0196305 |
| (conditioner | | | |
| resistant) | | | |
| Hair | GMPAMHWIHPFA | 119 | US 2006-0073111 |
| (shampoo | | | U.S. Pat. No. 7,285,264 |
| and | | | |
| conditioner | | | |
| resistant) | | | |
| Hair | HDHKNQKETHQRHAA | 120 | US 2006-0073111 |
| (shampoo | | | U.S. Pat. No. 7,285,264 |
| and | | | |
| conditioner | | | |
| resistant) | | | |
| Hair | HNHMQERYTDPQHSPSVNGL | 121 | US 2006-0073111 |
| (shampoo | | | U.S. Pat. No. 7,285,264 |
| and | | | |
| conditioner | | | |
| resistant) | | | |
| Hair | TAEIQSSKNPNPHPQRSWTN | 122 | US 2006-0073111 |
| (shampoo | | | U.S. Pat. No. 7,285,264 |
| and | | | |
| conditioner | | | |
| resistant) | | | |
| Hair | P-NTSQLST (hair-binding | 123 | US 2008-0175798 |
| (multiple | peptide)-GGG (spacer)- | | |
| binding | RTNAADHPKC (hair-binding | | |
| peptides) | peptide)-GGG (spacer)-NTSQLST | | |
| | (hair-binding peptide)-GGG | | |
| | (spacer)-RTNAADHPKC (hair- | | |
| | binding peptide)-GGG (spacer)- | | |
| | NTSQLST (hair-binding peptide)- | | |
| | GGG (spacer)-RTNAADHPKC | | |
| | (hair-binding peptide) | | |
| Hair | P-RTNAADHPAAVT (hair- | 124 | US 2008-0175798 |
| (multiple | binding peptide)-GGGCGGG | | |
| binding | (spacer)-RTNAADHPAAVT | | |
| peptides) | (hair-binding peptide)- | | |
| | GGGCGGG (spacer)- | | |
| | RTNAADHPAAVT (hair-binding | | |
| | peptide)-GGGC (spacer) | | |
| Hair | P-RTNAADHPAAVT (hair- | 125 | US 2008-0175798 |
| (multiple | binding peptide)-GGGCGGG | | |
| binding | (spacer)-IPWWNIRAPLNA (hair- | | |
| peptides) | binding peptide)-GGGCGGG | | |
| | (spacer)-DLTLPFH (hair-binding | | |
| | peptide)-GGGC (spacer) | | |
| Hair | P-RTNAADHP (hair-binding | 126 | US 2008-0175798 |
| (multiple | peptide)-GGG (spacer)- | | |
| binding | TPPELLHGDPRSKC (hair- | | |
| peptides) | binding peptide)-GGG (spacer)- | | |
| | RTNAADHP (hair-binding | | |
| | peptide)-GGG (spacer)- | | |
| | TPPELLHGDPRSKC (hair- | | |
| | binding peptide)-GGG (spacer)- | | |
| | RTNAADHP (hair-binding | | |
| | peptide)-GGG (spacer)- | | |
| | TPPELLHGDPRSKC (hair- | | |
| | binding peptide) | | |
| Hair | P-TPPTNVLMLATK (hair- | 127 | US 2008-0175798 |
| (multiple | binding peptide)-GGG (spacer)- | | |
| binding | RTNAADHPKC (hair-binding | | |
| peptides) | peptide)-GGG (spacer)- | | |
| | TPPTNVLMLATK (hair-binding | | |
| | peptide)-GGG (spacer)- | | |
| | RTNAADHPKC (hair-binding | | |
| | peptide)-GGG (spacer)- | | |
| | TPPTNVLMLATK (hair-binding | | |
| | peptide)-GGG (spacer)- | | |
| | RTNAADHPKC (hair-binding | | |
| | peptide) | | |
| Hair | P-RTNAADHP (hair-binding | 128 | US 2008-0175798 |
| (multiple | peptide)-GGG (spacer)- | | |
| binding | TPPTNVLMLATKKC (hair- | | |
| peptides) | binding peptide)-GGG (spacer)- | | |
| | RTNAADHP (hair-binding | | |
| | peptide)-GGG (spacer)- | | |
| | TPPTNVLMLATKKC (hair- | | |
| | binding peptide) GGG (spacer)- | | |
| | RTNAADHP (hair-binding | | |
| | peptide)-GGG (spacer)- | | |
| | TPPTNVLMLATKKC (hair- | | |
| | binding peptide) | | |
| Hair | PG (Spacer)-IPWWNIRAPLNA | 129 | US 2008-0175798 |
| (multiple | (hair-binding peptide)-GAG | | |
| binding | (spacer)- | | |
| peptides) | IPWWNIRAPLNA (hair-binding | | |
| | peptide)- | | |
| | GGSGPGSGG (spacer)- | | |
| | NTSQLST (hair-binding peptide)- | | |
| | GGG (spacer)- | | |
| | NTSQLST (hair-binding peptide)- | | |
| | GGPKK (spacer) | | |
| Hair and | KRGRHKRPKRHK | 130 | US 2007-0065387 |
| skin | | | US 2007-0110686 |
| (Empirical) | | | US 2007-0067924 |
| Hair and | RLLRLLR | 131 | US 2007-0065387 |
| skin | | | US 2007-0110686 |
| (Empirical) | | | |
| Hair and | HKPRGGRKKALH | 132 | US 2007-0065387 |
| skin | | | US 2007-0110686 |
| (Empirical) | | | |
| Hair and | KPRPPHGKKHRPKHRPKK | 133 | US 2007-0065387 |
| skin | | | US 2007-0110686 |
| (Empirical) | | | |
| Hair and | RGRPKKGHGKRPGHRARK | 134 | US 2007-0065387 |
| skin | | | US 2007-0110686 |
| (Empirical) | | | |
| Skin | TPFHSPENAPGS | 135 | U.S. Pat. No. 11/877,692 |
| | | | US 2005-0249682 |
| Skin | TPFHSPENAPGSK | 136 | US 2007-0110686 |
| Skin | TPFHSPENAPGSGGGS | 137 | US 2007-0110686 |
| Skin | TPFHSPENAPGSGGGSS | 138 | US 2007-0110686 |
| Skin | TPFHSPENAPGSGGG | 139 | US 2007-0110686 |
| Skin | FTQSLPR | 140 | U.S. Pat. No. 11/877,692 |
| | | | US 2005-0249682 |
| Skin | KQATFPPNPTAY | 141 | U.S. Pat. No. 11/877,692 |
| | | | US 2005-0249682 |
| | | | WO2004048399 |
| Skin | HGHMVSTSQLSI | 142 | U.S. Pat. No. 11/877,692 |
| | | | US 2005-0249682 |
| | | | WO2004048399 |
| Skin | LSPSRMK | 143 | U.S. Pat. No. 11/877,692 |
| | | | US 2005-0249682 |
| | | | WO2004048399 |
| Skin | LPIPRMK | 144 | US 2005-0249682 |
| | | | WO2004048399 |
| Skin | HQRPYLT | 145 | US 2005-0249682 |
| | | | WO2004048399 |
| Skin | FPPLLRL | 146 | US 2005-0249682 |
| | | | WO2004048399 |
| Skin | QATFMYN | 147 | WO2004048399 |
| Skin | VLTSQLPNHSM | 148 | WO2004048399 |
| Skin | HSTAYLT | 149 | WO2004048399 |
| Skin | APQQRPMKTFNT | 150 | WO2004048399 |
| Skin | APQQRPMKTVQY | 151 | WO2004048399 |
| Skin | PPWLDLL | 152 | WO2004048399 |
| Skin | PPWTFPL | 153 | WO2004048399 |
| Skin | SVTHLTS | 154 | WO2004048399 |
| Skin | VITRLTS | 155 | WO2004048399 |
| Skin | DLKPPLLALSKV | 156 | WO2004048399 |
| Skin | SHPSGALQEGTF | 157 | WO2004048399 |
| Skin | FPLTSKPSGACT | 158 | WO2004048399 |
| Skin | DLKPPLLALSKV | 159 | WO2004048399 |
| Skin | PLLALHS | 160 | WO2004048399 |
| Skin | VPISTQI | 161 | WO2004048399 |
| Skin | YAKQHYPISTFK | 162 | WO2004048399 |
| Skin | HSTAYLT | 163 | WO2004048399 |
| Skin | STAYLVAMSAAP | 164 | WO2004048399 |
| Skin (Body | SVSVGMKPSPRP | 165 | U.S. Pat. No. 11/877,692 |
| Wash | | | US 2006-0199206 |
| Resistant) | | | |
| Skin (Body | TMGFTAPRFPHY | 166 | U.S. Pat. No. 11/877,692 |
| Wash | | | US 2006-0199206 |
| Resistant) | | | |
| Skin (Body | NLQHSVGTSPVW | 167 | U.S. Pat. No. 11/877,692 |
| Wash | | | US 2006-0199206 |
| Resistant) | | | |
| Skin (Body | QLSYHAYPQANHHAP | 168 | U.S. Pat. No. 11/877,692 |
| Wash | | | US 2006-0199206 |
| Resistant) | | | |
| Skin (Body | NQAASITKRVPY | 169 | US 2006-0199206 |
| Wash | | | |
| Resistant) | | | |
| Skin (Body | SGCHLVYDNGFCDH | 170 | U.S. Pat. No. 11/877,692 |
| Wash | | | US 2006-0199206 |
| Resistant) | | | |
| Skin (Body | ASCPSASHADPCAH | 171 | U.S. Pat. No. 11/877,692 |
| Wash | | | US 2006-0199206 |
| Resistant) | | | |
| Skin (Body | NLCDSARDSPRCKV | 172 | U.S. Pat. No. 11/877,692 |
| Wash | | | US 2006-0199206 |
| Resistant) | | | |
| Skin (Body | NHSNWKTAADFL | 173 | U.S. Pat. No. 11/877,692 |
| Wash | | | US 2006-0199206 |
| Resistant) | | | |
| Skin (Body | GSSTVGRPLSYE | 174 | US 2006-0199206 |
| Wash | | | |
| Resistant) | | | |
| Skin (Body | SDTISRLHVSMT | 175 | U.S. Pat. No. 11/877,692 |
| Wash | | | US 2006-0199206 |
| Resistant) | | | |
| Skin (Body | SPLTVPYERKLL | 176 | US 2006-0199206 |
| Wash | | | |
| Resistant) | | | |
| Skin (Body | SPYPSWSTPAGR | 177 | U.S. Pat. No. 11/877,692 |
| Wash | | | US 2006-0199206 |
| Resistant) | | | |
| Skin (Body | VQPITNTRYEGG | 178 | US 2006-0199206 |
| Wash | | | |
| Resistant) | | | |
| Skin (Body | WPMHPEKGSRWS | 179 | US 2006-0199206 |
| Wash | | | |
| Resistant) | | | |
| Skin (Body | DACSGNGHPNNCDR | 180 | U.S. Pat. No. 11/877,692 |
| Wash | | | US 2006-0199206 |
| Resistant) | | | |
| Skin (Body | DHCLGRQLQPVCYP | 181 | US 2006-0199206 |
| Wash | | | |
| Resistant) | | | |
| Skin (Body | DWCDTIIPGRTCHG | 182 | U.S. Pat. No. 11/877,692 |
| Wash | | | US 2006-0199206 |
| Resistant) | | | |
| Fingernail | ALPRIANTWSPS | 183 | US 2005-0226839 |
| | | | U.S. Pat. No. 7,220,405 |
| Fingernail | YPSFSPTYRPAF | 184 | US 2005-0226839 |
| and Hair | | | U.S. Pat. No. 7,220,405 |

TABLE 1B

| Peptide ID | Structure | SEQ ID NO. |
|---|---|---|
| MEA4-biotin | HINKTNPHQGNHHSEKTQRQK-Biotin | 186 |
| Hair2-biotin | AQSQLPDKHSGLHERAPQRYK-Biotin | 187 |
| Hair4-biotin | TPPELAHTPHHLAQTRLTDRK-Biotin | 188 |
| HCP1-biotin | THSTHNHGSPRHTNADAGNPK-Biotin | 189 |

TABLE 1C

| Peptide ID | Sequence | SEQ ID NO: |
|---|---|---|
| Hair2Rnd1 | DRSKLYQSLEHRQPPGAHAQ | 190 |
| Gray3Rnd4 | AHDAKHEHRKQNHTQ | 191 |
| F4Rnd | QTHVSQPFLFHD | 192 |
| Rfe1 | WAPEKDHMQLMK | 193 |
| CA3 | NGNNHTDIPNRSSYTGGSFA | 194 |
| CA4 | SDETGPQIPHRRPTW | 195 |

TABLE 1D

| | | Binding | Part of | |
|---|---|---|---|---|
| Peptide | Sequence Description | Domain | affinity | SEQ ID |
| ID | (SEQ ID NO.) | Hair | pair | NO. |
| HC263 | MASGSDPS-Hair2- | HP2-Gray3 | HHHHHH | 196 |
| | GPEPEPEPEPIPEPPKEAPVVIEKPKPKPKPKPKPPA- | | (SEQ ID | |
| | Gray3-GSGGGGSP-HHHHHH | | NO: 235) | |
| HC264 | MASGSDPS-Hair2Rnd1- | HP2Randomized- | HHHHHH | 197 |
| | GPEPEPEPEPIPEPPKEAPVVIEKPKPKPKPKPKPPA- | Gray3Randomized | (SEQ ID | |
| | Gray3Rnd4-GSGGGGSP-HHHHHH | | NO: 235) | |
| HC214 | GSDPG-Gray3-GGG-Gray3-GGGGSGGGGSG- | Gray3- | HHHHHH | 198 |
| | HHHHHH | Gray3 | (SEQ ID | |
| | | | NO: 235) | |
| HC204 | GSDPG-F4Rnd-GGG-F4Rnd-GGGGSGGGGSG- | F4Rnd- | HHHHHH | 199 |
| | HHHHHH | F4Rnd | (SEQ ID | |
| | | | NO: 235) | |
| HC205 | GSDPG-IB5A-GGGGSGGGGSG-HHHHHH | IB5A | HHHHHH | 200 |
| | | | (SEQ ID | |
| | | | NO: 235) | |
| HC352 | MASGSDPS-Hair2- | HP2-Gray3 | none | 201 |
| | GPEPEPEPEPIPEPPKEAPVVIEKPKPKPKPKPKPPA- | | | |
| | Gray3-GSGGGGSP-Rfe1-GGAGGAG-Rfe1-GK | | | |
| HC423 | MASGSDPS-Hair2- | HP2-Gray3 | none | 202 |
| | GPEPEPEPEPIPEPPKEAPVVIEKPKPKPKPKPKPPA- | | | |
| | Gray3-GPGGGSGGGSGGGSGGGSPA-CA3- | | | |
| | GGAGGAG-CA3-GK | | | |
| HC424 | MASGSDPS-Hair2- | HP2-Gray3 | none | 203 |
| | GPEPEPEPEPIPEPPKEAPVVIEKPKPKPKPKPKPPA- | | | |
| | Gray3-GPGGGSGGGSGGGSGGGSPA-CA4- | | | |
| | GGAGGAG-CA4-GK | | | |

TABLE 1E

| | Description of Constructs | |
|---|---|---|
| Peptide ID | (Formula) | SEQ ID NO: |
| HCP5-dimer | HCP5-GGSGPGSGG-HCP5 | 204 |
| “(HCP5)2” | | |
| HCP5-dimer | HCP5-GGSGPGSGG-HCP5-K | 205 |
| c-terminal | | |
| lysine | | |
| SB33N | MLSENWLTNHPQN | 206 |
| HC260 | MASGSDPS-Hair2- | 207 |
| | GPEPEPEPEPIPEPPKEAPVVIEKPKPKP | |
| | KPKPKPPA-Gray3-GSGGGGSP-SB33N- | |
| | GGAGGAG-SB33N-GK | |

TABLE 1F

| | Binding Domain | Bridge | |
|---|---|---|---|
| Peptide ID | Formula of | Bridge | First part of Affinity |
| (SEQ ID NO:) | Hair Hand | (SEQ ID NO: 209) | Pair |
| HC634 | HP2E-TonB-Gray3A | (GSGGGGSP)₂ | (KQPN)₅ |
| (SEQ ID NO: 211) | | | (SEQ ID NO: 223) |
| HC635 | HP2E-TonB-Gray3A | (GSGGGGSP)₂ | (KQPN)₉ |
| (SEQ ID NO: 212) | | | (SEQ ID NO: 224) |
| HC636 | HP2E-TonB-Gray3A | (GSGGGGSP)₂ | (KQPN)₁₃ |
| (SEQ ID NO: 213) | | | (SEQ ID NO: 225) |
| HC637 | HP2E-TonB-Gray3A | (GSGGGGSP)₂ | (KQGPQ)₉ |
| (SEQ ID NO: 214) | | | (SEQ ID NO: 226) |
| HC638 | HP2E-TonB-Gray3A | (GSGGGGSP)₂ | (KQGPQG)₉ |
| (SEQ ID NO: 215) | | | (SEQ ID NO: 227) |
| HC639 | HP2E-TonB-Gray3A | (GSGGGGSP)₂ | (KGQGPQG)₉ |
| (SEQ ID NO: 216) | | | (SEQ ID NO: 228) |
| HC640 | HP2E-TonB-Gray3A | (GSGGGGSP)₂ | (KQPNHQPQ)₅ |
| (SEQ ID NO: 217) | | | (SEQ ID NO: 229) |
| HC641 | HP2E-TonB-Gray3A | (GSGGGGSP)₂ | (HQPNHQPQ)₅ |
| (SEQ ID NO: 218) | | | (SEQ ID NO: 230) |
| HC642 | HP2E-TonB-Gray3A | (GSGGGGSP)₂ | (HSPNHSPS)₅ |
| (SEQ ID NO: 219) | | | (SEQ ID NO: 231) |
| HC643 | HP2E-TonB-Gray3A | (GSGGGGSP)₂ | HTSPQHHTSPHPHKNPQ |
| (SEQ ID NO: 220) | | | HTSPTPN |
| | | | (SEQ ID NO: 232) |
| HC644 | HP2E-TonB-Gray3A | (GSGGGGSP)₂ | HTKQNHTSPTPHHTNSP |
| (SEQ ID NO: 221) | | | QHTSPHP |
| | | | (SEQ ID NO: 233) |
| HC645 | HP2E-TonB-Gray3A | (GSGGGGSP)₂ | HNNRQHTSPHPQHTKQN |
| (SEQ ID NO: 222) | | | HTSPTPH |
| | | | (SEQ ID NO: 234) |

In one embodiment, the peptidic binding domain comprises at least one peptide set forth in the group consisting of SEQ ID NOs: 1-184, 186-189, 196-200, 204-205, and 211-222. In another embodiment, the peptidic binding domain includes a hair-binding peptide selected from the group consisting of SEQ ID NOs: 1-134, 184, 186-189, 196-200, and 211-222. In yet another embodiment, the peptidic binding domain is a skin-binding selected from the group consisting of SEQ ID NOs: 130-182. In other embodiments, the peptidic binding domain comprises at least one of SEQ ID NOs 196-200 or SEQ ID NOs. 210-222.

In yet another embodiment, the peptidic binding domain comprises at least one skin-binding peptide selected from the group consisting of SEQ ID NOs: 130-182. In still another embodiment, the peptidic binding domain is nail-binding selected from the group consisting of SEQ ID NOs: 183-184.

In addition to comprising at least one binding domain that binds to at least one of human hair, human skin, or human nail, the peptidic components of the invention further comprise the first part of an affinity pair. As used herein, “affinity pair” refers to a pair of agents having a known affinity for each other wherein the first part of the affinity pair was not derived from biopanning. Affinity pairs will be based on bonding associations that are not covalent bond-based, including, for example, ionic bond-based (electrostatic interaction), hydrogen bond-based, hydrophobic bond-based, chelation-based, biological affinity-based, or the affinity pair is based on a combination thereof.

For example, affinity pairs of the invention may include ionic-bond pairs. “Ionic bond” pairs refers to an association complex of two moieties wherein one has a net positive charge and the other has a net negative charge. Ionic bonds, also referred to as electrostatic interactions, are among the strongest bonds, comparable in strength to covalent bonds, and are of long-range, on the order of 50 nm. (Isrealachvili, J. N., Intermolecular and Surface Forces, 2nd ed.; Academic Press: New York, N.Y. (1992) pp. 32-34).

Certain amino acids contain ionizable side groups, for example, the carboxyl groups in the side chains of aspartic and glutamic acids and the amino groups located at lysine, arginine and histidine residues. Some peptides often contain net charges (positive or negative) and certain charge distributions when any of the charged amino acids are in the peptide sequence. The net charges of the whole or portion of peptide molecule can induce electrostatic attraction with the oppositely charged second part of the affinity pair on the benefit agent, or induce electrostatic repulsion with the similarly charged second part of the affinity pair on the benefit agent.

Examples of ionic (electrostatic) binding pairs include, but are not limited to, negative charged peptides coupled to positively charged particulate benefit agents, negative charged peptides coupled to particulate benefit agents comprised of or coated with a positively charged coating (e.g., anion exchange resins), positively charged peptides coupled to negatively charged particulate benefit agents (e.g., mica, silica), positively charged peptides couple to particulate benefit agents comprising a coating that provides a negative charge (e.g., cationic exchange resins having groups such as SO₄⁻²).

The charge and the charge density on the second part of the affinity pair on the particulate benefit agent can be obtained and regulated with proper surface treatments and pH conditions. Charges could originate from: 1) ionization of surface functional groups such as amino, carboxyl, sulfonic, and hydroxyl groups etc; 2) specific adsorption of ions from solutions. In this context, “specific adsorption” implies that the adsorption is partly of non-electric nature so that the adsorbed ions can create net surface charges. For inert benefit agents, multiple surface treatment approaches in the art could be used to create ionizable functional groups: 1) using oxygen plasma to oxidize the surface or plasma polymerization of specialty gas to create surface hydroxyl and other groups (C. L. Rinsch et al, Langmuir (1996), 12 (2995-3002); 2) forming self-assembled monolayers with terminal function groups, such as aminopropyl silane forming siloxane monolayers on metal oxide surface (Xia, Y. N., and Whitesides, G. M., Angew. Chem. Int. Ed. (1998), 37:551-575); 3) using layer-by-layer assembly process to adsorb polyelectrolyte multiplayer onto any surfaces to give charged surface with desired charge sign and charge density (Decher, G., Science (1997), 277:1232-1237); and 4) precipitation-coating of charged polymers or sol-gel. Surface charges of the benefit agents could be characterized by its surface isoelectric point (IEP), the pH value at which the net surface charges are zero. So at pH lower than its IEP, the benefit agent, in particular the second part of the affinity pair on the particulate benefit agent, bears positive charges; while at pH greater than its IEP, the benefit agent bears negative charges.

Electrostatic interaction ranges can be further modulated with ionic strength: lower ionic strength provides longer interaction range, while higher ionic strength provides shorter interaction range. The modulation of the interaction range can be applied to obtaining stable peptide-benefit agent adduct at low ionic strength, but enhancing benefit agent deliver to body surfaces at higher ionic strength.

The net charge of the first part of the affinity pair may be negative or positive depending upon the pH of the system. In one embodiment, the net charge of the first part of the affinity pair is positive at a specified pH wherein the pH may range from 3.0 to about 10. In another embodiment, the net charge of the first part of the affinity pair is negative at a specified pH wherein the pH may range from 3.0 to about 10.

Affinity pairs of the invention may also include hydrogen-bond-based pairs. “Hydrogen-bond” pairs refers to an association complex of the hydrogen atom of a relatively electronegative atom of one moiety with an electronegative atom of the other moiety.

“Hydrophobic-bond” pairs refers to an association complex of two moieties in which both moieties have hydrophobic domains or characteristics that enable them to form an associative complex. The surface of particulate benefit agent may be hydrophobic. As such, one may incorporate into the peptide component a first part of the affinity pair that comprises an effective number of hydrophobic amino acid residues. The surface of the particulate benefit agent may inherently be hydrophobic or may be modified to have a hydrophobic surface capable of associating with another hydrophobic moiety. For example, the particulate benefit agent may be coated with a hydrophobic polymer using any number of well known coating techniques. A first part of the affinity pair that includes a hydrophobic peptide will typically be comprised of hydrophobic amino acids having a hydropathy index of at least 1.5 (Kyte and Doolittle, J. Mol. Biol. (1982) 157(157): 105-132). In one embodiment, the hydrophobic amino acids are selected from the group consisting of isoleucine, valine, leucine, phenylalanine, cysteine, methionine, and alanine.

“Chelation-based” pairs refers to a coordinate covalent bonding complex of a Lewis acid and a Lewis base where the Lewis base donates two or more lone pairs of electrons to the Lewis acid. An example of chelation-based pairs is the interaction of various amino acid side chains and metal ions. Exemplary metals for use in chelation-based pairs include divalent metals, for example, nickel, copper, cobalt, and zinc.

“Polyhistidine tags” are often used to bind to immobilized metal ions such as nickel, copper, cobalt, or zinc. The metal ion is typically incorporated into media such as nitriloacetic acid (NTA)-agarose, HisPur cobalt resin, iminodiacetic acid (IDA) resin, carboxylmethylaspartate (CMA) resin, TALON® (or any other immobilized metal affinity chromatography (IMAC) resin IMAC). Metal affinity resins are commercially available from various vendors such as Thermo Fisher Scientific (Rockford, Ill.), EMD BioSciences (Madison, Wis.), and Clontech (Palo Alto, Calif.). The polyhistidine tag may be synthetic or a naturally-occurring histidine affinity tag such as “HAT” (KDHLIHNVHKEFHAHAHNK (SEQ ID NO: 185)). In one embodiment, the polyhistidine tag ranges from 6 to about 10, 6 about 8, or about 6 consecutive (“HHHHHH”) histidine residues in length.

In one embodiment, the peptidic component comprises at least one polyhistidine tag capable of binding to an immobilized metal ion on the surface of the particulate benefit agent. In another embodiment, the particulate benefit agent comprises an effective amount of an appropriate media on the surface of the particle. The metal chelate resin may be applied as a partial or complete coating on the surface of the particulate benefit agent. In another embodiment, the resin applied to the surface of the particulate benefit agent comprises a tetradentate metal chelator (U.S. Pat. No. 5,962,641; herein incorporated by reference). In another embodiment, the affinity pair is a chelation pair that includes a polyhistidine-tag, i.e., an amino acid motif incorporated that consists of an effective number of histidine residues capable of binding to a resin immobilized metal ion with micromolar affinity, incorporated into the peptidic component and a metal ion incorporated into the particulate benefit agent, wherein the metal ion selected from the group consisting of nickel, copper, cobalt, zinc, and mixtures thereof.

Other examples of affinity pairs of the invention include, but are not limited to biotin: avidin, biotin:streptavidin, streptavidin tags:streptavidin, maltose binding protein (MBP):maltose or amylase, glutathione S-transferase (GST):glutathione.

Affinity pairs can also be biological affinity-based. As used herein, biological affinity does not encompass antibody-antigen affinity. Antibody-antigen affinity is specifically excluded from the scope of the invention. In one embodiment, the affinity pair may include an epitope tag:antibody pair wherein the peptide-based reagent comprises the epitope sequence and the particulate benefit agent comprises the corresponding antibody. Examples of commercially epitope tags include, but are not limited to HA-tag, FLAG-tag, E-tag, S-tag, and myc-tag.

In certain embodiments of the invention, the first part of the affinity pair is selected from the group consisting of a polyhistidine tag, biotin, a streptavidin tag, maltose binding protein, glutathione S-transferase, an epitope tag, HA-tag, FLAG-tag, E-tag, S-tag, myc-tag, and SEQ ID NOs: 185, 206, and 223-234. In another embodiment, the first part of the affinity pair is selected from the group consisting of a polyhistidine tag, biotin, and SEQ ID NOs: 185, 206, and 223-234.

Optionally, the binding domains of the present invention can exhibit preferential binding for at least one of human hair, skin, or nail over other materials. For example, the binding domains of the invention may further preferentially bind to human hair, skin or nail over wool, cashmere, or yak hair. In another embodiment, the binding domains of the invention further preferentially bind to human hair, skin or nail over cotton or modified cellulosic fiber. In yet another embodiment, the binding domains of the invention further preferentially bind to human hair, skin or nail over metal, ceramic, porcelain, glass, silk, wood, polyester, or polyvinylchloride.

### The Benefit Agent

“Benefit agent,” as that term is used in the present invention, is directed to cosmetic compositions containing compositions or agents with properties that impart benefits to human hair, skin, and/or nail when deposited thereon. Benefit agents of the invention are in particulate form, i.e., the benefit agent is provided as small, discrete particles. The particulate benefit agents of the present invention have the second part of the affinity pair and are incorporated into a stable particulate dispersion having average particle size of between about 0.01 micron (10 nm) and about 75 microns (75,000 nm). In one embodiment, the average particle size is 0.01 micron to 75 microns, as measured by a light scattering method such as laser diffraction and/or dynamic light scattering. In some embodiments, the average particle size is less than about 60 microns, less than about 40 microns, or less than about 10 microns. In other embodiments, the average particle size is between about 0.2 microns (200 nm) and 0.4 microns (400 nm). In other embodiments, the particles of the dispersion will be nanoparticles, i.e., will have average particle size of between about 10 nm and about 100 nm.

It will be understood by those skilled in the art that the “particle size” referenced herein will refer to the particle size measurements obtained using a light scattering methods such as laser diffraction (see ISO 13320-1:1996; International Organization for Standards, Geneva, Switzerland) and/or dynamic light scattering (see ISO 13321:1996) methodologies, both of which are known in the art. Exemplary systems are available from Malvern Instruments Ltd. Worcestershire, United Kingdom.

It has been discovered that providing the particulate benefit agent in a stable particulate dispersion having average particle size of between about 0.01 micron and about 75 microns facilitates the coupling of the benefit agent to the peptidic component of the cosmetic systems of the invention.

“Stable dispersions of particulate benefit agent” of the present invention, refers to particulate benefit agent particles dispersed within a sample matrix that is stable over time.

As used herein, the term “stable” will refer to dispersions wherein particles dispersed within a sample matrix are stable over time. Particles will be considered stably dispersed when the average particle size of a sample remains fairly constant with time. In one embodiment, a sample is stably-dispersed if the average particle size of the sample does not increase by more than 100% over the initial particle size of the particulate benefit agent within 24 hours after dispersion formation. In another embodiment, the sample may be stably-dispersed if the average particle size of the sample does not increase by more than 50% over the initial particle size of the particulate benefit agent within 2 days after dispersion formation. In certain embodiments, there is no more than a 50% increase in average particle size within 3 days after dispersion formation. In other embodiment, there is no more than a 50% increase in average particle size within at least 5 days of dispersion formation. In still other embodiments, there is no more than a 50% increase in average particle size within 7 days of dispersion formation. In one embodiment, a particulate dispersion is stable when the average particle size does not increase more than 50% over at least 7 days, without the detection of any agglomerates larger than 50 primary particles. One of skill in the art will recognize that stable particle dispersions may have some settling over time, so long as the particles can be re-dispersed easily with a minimal amount of energy (e.g. gentle manual shaking/agitation that is typically associated with manual mixing/shaking to reform a uniform dispersion of particles within the cosmetic composition or cosmetic system).

Means to form stable particle dispersions have been reported in the art including, but not limited to the use of sterically stabilized dispersions, dispersants, ionic dispersants, non-ionic dispersants, and polymeric dispersants, to name a few.

Polymeric dispersants are widely used to stabilize pigments in coating systems such as paints and finishes, and in ink jet printing inks (Reuter et al., Progress in Organic Coatings 37:161 167 (1999), Schmitz et al, Progress in Organic Coatings 35:191 196 (1999), and Spinelli, Adv. Mater. 10:1215 1218 (1998)). The dispersant serves to form a shell around the pigment particle, i.e., the particulate benefit agent, preventing flocculation and coagulation. In aqueous systems, the pigment dispersion is generally stabilized by either a nonionic or ionic technique. In the non-ionic technique, the pigment particles are stabilized by a polymer that has a water-soluble, hydrophilic section that extends into the water and provides entropic or steric stabilization. Representative polymers useful for this purpose include polyvinyl alcohol, cellulosics, and ethylene oxide modified phenols. While the non-ionic technique is not sensitive to pH changes or ionic contamination, it has a major disadvantage for many applications in that the final product is water sensitive. Thus, if used in ink applications or the like, the pigment will tend to smear upon exposure to moisture.

In the ionic technique, the pigment particles are stabilized by a polymer of an ion containing monomer, such as neutralized acrylic, maleic, or vinyl sulfonic acid. The polymer provides stabilization through a charged double layer mechanism whereby ionic repulsion hinders the particles from flocculation. Since the neutralizing component tends to evaporate after application, the polymer then has reduced water solubility and the final product is not water sensitive. Polymer dispersants, such as block and graft polymers, that provide both steric and ionic stabilization make the most robust pigment dispersions (Spinelli, supra).

Polymer dispersants having both random and block structures have been disclosed. For example, Ohta et al. in U.S. Pat. No. 4,597,794 disclose a random polymer dispersant having ionic hydrophilic segments and aromatic hydrophobic segments that adhere to the pigment surface. Ma et al. in U.S. Pat. No. 5,085,698 disclose the use of AB or BAB block copolymers as dispersants for aqueous ink jet inks. The A segment is a hydrophobic homopolymer or copolymer that serves to bind to the pigment particle and the B segment is a hydrophilic polymer, or salt thereof, that serves to disperse the pigment in the aqueous medium. Ma et al. in U.S. Pat. No. 5,519,085 disclose an ABC triblock polymer dispersant, wherein the A segment is a hydrophilic polymer that serves to facilitate dispersion of the pigment in water, the B segment is a polymer capable of binding to the pigment, and the C segment is a hydrophilic or hydrophobic polymer that serves to stabilize the dispersion. A combination of polymer dispersants may also be used, as described by Rose et al. in GB 2349153. While these random and block polymer dispersants offer good stability for the dispersed pigment, further improvements are desired for more high quality coating applications. For example, dispersants having a stronger interaction with the pigment would improve the stability of the dispersion. Moreover, a dispersant with a stronger interaction with the coating substrate would result in a more durable coating. This is particularly important for textile printing where enhanced durability is required.

A self-dispersing pigment is a pigment that has been surface modified with chemically attached, dispersibility imparting groups to allow stable dispersion without a separate dispersant. For dispersion in an aqueous carrier medium, surface modification involves addition of hydrophilic groups and most typically ionizable hydrophilic groups. The self-dispersing pigment may be prepared by grafting a functional group or a molecule containing a functional group onto the surface of the pigment, by physical treatment (such as vacuum plasma), or by chemical treatment (for example, oxidation with ozone, hypochlorous acid or the like). A single type or a plurality of types of hydrophilic functional groups may be bonded to one pigment particle. Self-dispersing pigments are described, for example, in U.S. Pat. No. 5,571,311, U.S. Pat. No. 5,609,671, U.S. Pat. No. 5,968,243, U.S. Pat. No. 5,928,419, U.S. Pat. No. 6,323,257, U.S. Pat. No. 5,554,739, U.S. Pat. No. 5,672,198, U.S. Pat. No. 5,698,016, U.S. Pat. No. 5,718,746, U.S. Pat. No. 5,749,950, U.S. Pat. No. 5,803,959, U.S. Pat. No. 5,837,045, U.S. Pat. No. 5,846,307, U.S. Pat. No. 5,895,522, U.S. Pat. No. 5,922,118, U.S. Pat. No. 6,123,759, U.S. Pat. No. 6,221,142, U.S. Pat. No. 6,221,143, U.S. Pat. No. 6,281,267, U.S. Pat. No. 6,329,446, U.S. Pat. No. 6,332,919, U.S. Pat. No. 6,375,317, U.S. Pat. No. 6,287,374, U.S. Pat. No. 6,398,858, U.S. Pat. No. 6,402,825, U.S. Pat. No. 6,468,342, U.S. Pat. No. 6,503,311, U.S. Pat. No. 6,506,245, and U.S. Pat. No. 6,852,156. The disclosures of the preceding references are incorporated by herein by reference.

The zeta potential indicates the degree of repulsion between adjacent, similarly charged particles in a dispersion. Colloids with high zeta potential (negative or positive) are electrically stabilized while colloids with low zeta potentials tend to coagulate or flocculate (“Zeta Potential of Colloids in Water and Waste Water”, ASTM Standard D 4187-82, American Society for Testing and Materials, 1985). In one embodiment, the absolute value of the zeta potential of the particulate benefit agent is at least 25 mV. In another embodiment, the absolute value of the zeta potential of the peptide reagent-particulate benefit agent complex is at least 25 mV.

The second part of the affinity part may be incorporated into the benefit agent in any number of ways. For example, the physical properties of the benefit agent may include the second part of the affinity pair. The second part of the affinity pair may be inherently present in the benefit agent or the benefit agent can be modified to include the second part of the affinity pair. For instance, in some embodiments, the benefit agent may be a charged colored pigment or dye, the charge forming the second part of an ionic affinity pair. The second part of the affinity pair may be an applied material or coating (e.g., a metal chelate resin) that has affinity for the first member of the affinity pair (e.g., a polyhistidine tag). In other embodiments, the second part of the affinity pair may be covalently attached to the benefit agent.

Non-limiting examples of particulate benefit agents useful in the present invention include sunscreen agents, antimicrobial agents, sparkling particles, odor-control agents, conditioning agents, anti-fungal agents, fragrances, anti-lyses agents, aromatherapy agents, insect repellent agents, and the like.

Non-limiting examples of sunscreen agents include inorganic particulates, such as zinc oxide and titanium dioxide; and organic particulates, such as methylene bis-benzotriazolyl tetramethylbutylphenol (available as Bisoctrizole from Ciba Specialty Chemicals of Basel, Switzerland). Non-limiting examples of particulate antimicrobial agents include silver-based particles and activated carbon-based particles. Examples of microspheres containing particulate benefit agents may include encapsulated or microencapsulated benefit agents, which retain the benefit agent within the encapsulation during application and allow the benefit agent to be released from the encapsulation at some desired time after deposition on the keratin-containing surface. Examples of odor-control agents include activated carbon particles and zeolites.

The benefit agents of this invention may also be colored particulates, including colored pigments, colored particles, such as microparticles or nanoparticles, or combinations of these.

Pigments, particularly metal compounds or semimetallic compounds, may be used in the compositions and methods of this invention in ionic, nonionic or oxidized form. The pigments may be in this form either individually or in admixture or as individual mixed oxides or mixtures thereof, including mixtures of mixed oxides and pure oxides. Examples are the titanium oxides (for example TiO₂), zinc oxides (for example ZnO), aluminum oxides (for example Al₂O₃), iron oxides (for example Fe₂O₃), manganese oxides (for example MnO), silicon oxides (for example SiO₂), silicates, cerium oxide, zirconium oxides (for example ZrO₂), barium sulfate (BaSO₄) or mixtures thereof and the like. Suitable pigments are commercially available. An example is Hombitec® L5 (INCI name: titanium dioxides) supplied by Merck.

Other examples of pigments include the following: D&C Red No. 36, D&C Red No. 30, D&C Orange No. 17, Green 3 Lake, Ext. Yellow 7 Lake, Orange 4 Lake, Red 28 Lake, the calcium lakes of D&C Red Nos. 7, 11, 31 and 34, the barium lake of D&C Red No. 12, the strontium lake D&C Red No. 13, the aluminum lakes of FD&C Yellow No. 5 and No. 6, the aluminum lakes of FD&C No. 40, the aluminum lakes of D&C Red Nos. 21, 22, 27, and 28, the aluminum lakes of FD&C Blue No. 1, the aluminum lakes of D&C Orange No. 5, the aluminum lakes of D&C Yellow No. 10; the zirconium lake of D&C Red No. 33, CROMOPHTHAL® Yellow, SUNFAST® Magenta, SUNFAST® Blue, iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, titanium dioxide nanoparticles, zinc oxide, barium oxide, ultramarine blue, bismuth citrate, hydroxyapatite, zirconium silicate, carbon black particles and the like.

The pigments or particles of this invention can be coated or uncoated, and coated particles can be anionic, hydrophilic, or hydrophobic. Suitable anionic coatings include, for example, silica, aluminosilicate, sodium C14-16 olefin sulfonate, disodium stearoyl glutamate, sodium stearoyl glutamate/sodium trideceth-6 carboxylate, and sodium polyacrylates/hydrogenated lecithin/aluminum hydroxide. Examples of uncoated pigments suitable for use in the present invention are given in Table 2.

TABLE 2
Uncoated Pigments

| Pigment Name | Chemical Name | Surface Coating | Source |
|---|---|---|---|
| SOFT TEX BLACK | Iron Oxide | Uncoated | Sun Chemical |
| | | | Corporation |
| | | | Cincinnati OH |
| SUNCROMA RED | Iron Oxide | Uncoated needle shape | Sun Chemical |
| | | | Corporation |
| | | | Cincinnati OH |
| SOFT TEX RED | Iron Oxide | Uncoated | Sun Chemical |
| | | | Corporation |
| | | | Cincinnati OH |
| NANOPHASE NANOARC COSMETIC | Iron Oxide | Uncoated | Nanophase |
| IRON OXIDE (RED) | | | Technologies |
| | | | Corporation, |
| | | | Romeoville, IL |
| NANOPHASE NANOARC BLACK | Iron Oxide | Uncoated | Nanophase |
| IRON OXIDE | | | Technologies |
| | | | Corporation, |
| | | | Romeoville, IL |
| SENSIENT UNIPURE RED LC381 EM | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE BLACK LC989 | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE YELLOW LC 182 | Iron Oxide | Uncoated | LCW - Sensient |
| EM | | | Cosmetic |
| | | | Technologies |
| | | | Saint Ouen |
| | | | L'Aumone, |
| | | | France |
| SENSIENT UNIPURE RED LC383 EM | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE RED LC386 EM | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE WHITE LC 981 | Titanium Dioxide | Uncoated | LCW - Sensient |
| EM | | | Cosmetic |
| | | | Technologies |
| | | | Saint Ouen |
| | | | L'Aumone, |
| | | | France |
| SENSIENT UNIPURE YELLOW LC 181 | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE YELLOW LC 380 | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE BROWN LC881 | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE BROWN LC887 | Iron Oxide | Uncoated | LCW - Sensient |
| | | | Cosmetic |
| | | | Technologies |
| | | | Saint Ouen |
| | | | L'Aumone, |
| | | | France |
| SENSIENT UNIPURE BROWN LC889 | Iron Oxide | Uncoated | LCW - Sensient |
| | | | Cosmetic |
| | | | Technologies |
| | | | Saint Ouen |
| | | | L'Aumone, |
| | | | France |
| KOBO TAROX IROX TRR-100 | Iron Oxide | Uncoated | Kobo Products, |
| | | | Inc. South |
| | | | Plainfield NJ |
| KOBO BLACK NF | Iron Oxide | Uncoated | Kobo Products, |
| | | | Inc. South |
| | | | Plainfield NJ |

Examples of anionic coated pigments are given in Table 3.

TABLE 3
Anionic Coated Pigments

| Pigment Name | Chemical Name | Surface Coating | Source |
|---|---|---|---|
| SYMPHOLIGHT RW | Iron Oxide | silica treated | Presperse, Inc. |
| | | | Somerset, NJ |
| SYMPHOLIGHT BW | Iron Oxide | silica treated | Presperse, Inc. |
| | | | Somerset, NJ |
| SP-4405 Surface Passivated Black Oxide | Iron Oxide | aluminosilicate | Color |
| | | | Techniques, Inc., |
| | | | South Plainfield |
| | | | NJ |
| SP-Surface Passivated titanium dioxide | Titanium Dioxide | aluminosilicate | Color |
| | | | Techniques, Inc., |
| | | | South Plainfield |
| | | | NJ |
| SINERT BP-10 | Iron Oxide | silica | Kobo Products, |
| | | | Inc. South |
| | | | Plainfield NJ |
| SINERT RP5-10 | Iron Oxide | silica | Kobo Products, |
| | | | Inc. South |
| | | | Plainfield NJ |
| BRO-C5 | Iron Oxide | silica | Kobo Products, |
| | | | Inc. South |
| | | | Plainfield NJ |
| AQUASPERSABIL RIO | Iron Oxide | sodium C₁₄₋₁₆ Olefin | Presperse, Inc. |
| | | Sulfonate | Somerset, NJ |
| AMINO ACID (NAID) TREATED IRON | Iron Oxide | Disodium Stearoyl | U.S. Cosmetics |
| OXIDE | | Glutamate | Corporation, |
| | | | Dayville CT |
| BLACK BL-100 SPA | Iron Oxide | Sodium Stearoyl | Kobo Products, |
| | | Glutamate (And) Sodium | Inc. South |
| | | Trideceth-6 Carboxylate | Plainfield NJ |
| PALI TREATED IRON OXIDE | Iron Oxide | Sodium | U.S. Cosmetics |
| | | Polyacrylates/Hydrogenated | Corporation, |
| | | Lecithin/Aluminum | Dayville CT |
| | | Hydroxide | |

Examples of hydrophilic coated pigments are given in Table 4.

TABLE 4
Hydrophilic Coated Pigments

| | Chemical | | |
|---|---|---|---|
| Pigment Name | Name | Surface Coating | Source |
| CT-2 Black | Iron Oxide | chitosan | Kobo Products, |
| BL-100P | | | Inc. South |
| | | | Plainfield NJ |
| CT-2 Red R--516P | Iron Oxide | chitosan | Kobo Products, |
| | | | Inc. South |
| | | | Plainfield NJ |
| BGRO-SW2 | Iron Oxide | PEG-8 Methyl Ether | Kobo Products, |
| | | Triethoxysilane | Inc. South |
| | | | Plainfield NJ |
| GA-7403 | Iron Oxide | Galactoarabinan | Color |
| Galactoarabinan | | | Techniques, Inc., |
| treated black | | | South Plainfield |
| oxide | | | NJ |
| Lauroyl Lysine | Iron Oxide | Lauroyl Lysine | Color |
| treated | | | Techniques, Inc., |
| iron oxide | | | South Plainfield |
| | | | NJ |
| C2-5 Black BL-100 | Iron Oxide | cellulose | Kobo Products, |
| | | | Inc. South |
| | | | Plainfield NJ |
| C2-5 Red R-516L | Iron Oxide | cellulose | Kobo Products, |
| | | | Inc. South |
| | | | Plainfield NJ |

Examples of hydrophobic coated pigments are given in Table 5.

TABLE 5

| Hydrophobic Coated Pigments | | | |
|---|---|---|---|
| Pigment Name | Chemical Name | Surface Coating | Source |
| BBO-I2 | Iron Oxide | Isopropyl Titanium | Kobo Products, |
| | | Triisostearate | Inc. South |
| | | | Plainfield NJ |
| ASC-2 Black BL-100 | Iron Oxide | Acrylates/Dimethicone | Kobo Products, |
| | | Copolymer | Inc. South |
| | | | Plainfield NJ |
| BBO/MM1.5 | Iron Oxide | Magnesium Myristate | Kobo Products, |
| | | | Inc. South |
| | | | Plainfield NJ |
| BBO-11S2 | Iron Oxide | Triethoxy Caprylylsilane | Kobo Products, |
| | | | Inc. South |
| | | | Plainfield NJ |
| ASC-2 BLACK BL-100 | Iron Oxide | Acrylates/Dimethicone | Kobo Products, |
| | | Copolymer | Inc. South |
| | | | Plainfield NJ |
| PF-5 BLACK BL-100 | Iron Oxide | C9-15 Fluoroalcohol | Kobo Products, |
| | | Phosphates | Inc. South |
| | | | Plainfield NJ |
| PF-10 STT-65C-S | titanium dioxide | C9-15 Fluoroalcohol | Kobo Products, |
| | | Phosphates (And) | Inc. South |
| | | Triethanolamine | Plainfield NJ |
| PF-5 SI01-2 TiO2 CR-50 | titanium dioxide | C9-15 Fluoroalcohol | Kobo Products, |
| | | Phosphates (And) | Inc. South |
| | | Methicone (And) Alumina | Plainfield NJ |
| FC2-55 Black BL-100 | Iron Oxide | C9-15 Fluoroalcohol | Kobo Products, |
| | | Phosphate (And) | Inc. South |
| | | Microcrystalline Cellulose | Plainfield NJ |
| BGRO-BAS2 | Iron Oxide | Triethoxysilylethyl | Kobo Products, |
| | | Polydimethylsiloxyethyl | Inc. South |
| | | Hexyl Dimethicone | Plainfield NJ |
| BGRO-TTS2 | Iron Oxide | Isopropyl Titanium | Kobo Products, |
| | | Triisostearate/ | Inc. South |
| | | Triethoxycaprylylsilane | Plainfield NJ |
| | | Crosspolymer | |
| BGBO-TTM2 | Iron Oxide | Isopropyl titanium | Kobo Products, |
| | | triisostearate/Methicone | Inc. South |
| | | Crosspolymer | Plainfield NJ |
| PF-5 BLACK BL-100 | Iron Oxide | C9-15 Fluoroalcohol | Kobo Products, |
| | | Phosphates | Inc. South |
| | | | Plainfield NJ |
| AS-5123 alkyl silane treated red iron oxide | Iron Oxide | Triethoxycaprylylsilane | Color |
| | | | Techniques, Inc., |
| | | | South Plainfield |
| | | | NJ |
| DI-9825 dimethicon treated Micro TiO2 | Titanium Dioxide | dimethicone | Color |
| | | | Techniques, Inc., |
| | | | South Plainfield |
| | | | NJ |
| PFD-8146 Fluoropropyl Methicone treated | Iron Oxide | Fluoropropyl Methicone | Color |
| black oxide | | | Techniques, Inc., |
| | | | South Plainfield |
| | | | NJ |
| DL-9146 Hydrophobic Black Iron Oxide | Iron Oxide | methicone | Color |
| | | | Techniques, Inc., |
| | | | South Plainfield |
| | | | NJ |
| MM-4146 Magnesium Myristate/I.O. | Iron Oxide | Magnesium Myristate | Color |
| Black | | | Techniques, Inc., |
| | | | South Plainfield |
| | | | NJ |
| Oleosperse RIO | Iron Oxide | Dimethicone | Presperse, Inc. |
| | | | Somerset, NJ |
| Lecithin (LT/VLI) Treated Iron Oxide | Iron Oxide | Hydrogenated Lecithin | U.S. Cosmetics |
| | | | Corporation, |
| | | | Dayville CT |
| Lipoamino Acid Treated Iron Oxide | Iron Oxide | Palmitoyl Proline/ | U.S. Cosmetics |
| | | Magnesium Palmitoyl | Corporation, |
| | | Glutamate/Sodium | Dayville CT |
| | | Palmitoyl Sarcosinate/ | |
| | | Palmitic Acid | |
| Metal Soap (MT/MPT) Treated Iron Oxide | Iron Oxide | Aluminum Dimyristate | U.S. Cosmetics |
| | | | Corporation, |
| | | | Dayville CT |
| Dimethicone (SAT) Treated Iron Oxide | Iron Oxide | Dimethicone | U.S. Cosmetics |
| | | | Corporation, |
| | | | Dayville CT |
| Dimethicone/Glyceryl Rosinate (ST- | Iron Oxide | Dimethicone/Glyceryl | U.S. Cosmetics |
| PEG/MOD) Treated Iron Oxide | | Rosinate | Corporation, |
| | | | Dayville CT |

### The Cosmetic System

An exemplary method of using the cosmetic systems of the invention comprises the application of the peptidic component to at least one of human hair, skin, or nail. After a period of time sufficient for the peptidic component to bind to the hair, skin, or nail, the stable dispersion of particulate benefit agent is applied to peptidic component bound to hair, skin, or nail. The stable dispersion should be applied for a period of time sufficient for the first part of the affinity pair of the peptidic component to couple to the second part of the affinity pair of the benefit agent.

Once the benefit agent and peptidic component are coupled, the affinity pair can be disrupted, resulting in the uncoupling of the peptidic component and the benefit agent. Reagents capable of disrupting the affinity pair will be based on the type of affinity pair used in the cosmetic system. Typical of such reagents are aqueous solutions including buffers having high or low pH or high or low ionic strength, as required.

Certain methods of applying a benefit agent to at least one of human hair, human skin, or human nail comprise contacting the human hair, skin, or nail with a composition comprising a peptidic component having at least one binding domain which binds to at least one of the human hair, skin, or nail with a K_{d} or MB₅₀ value of 10⁻⁵ molar or less, and which further comprises the first part of an affinity pair, for a time sufficient for the binding domain to bind to the human hair, skin, or nail; and subsequently contacting the human hair, skin, or nail with a stable dispersion of particulate benefit agent having average particle size of between about 0.01 micron to about 75 microns and the second part of the affinity pair; wherein the binding domain has a greater binding affinity for the human hair, skin, or nail than it has for the particles of the dispersion.

Other methods include removing a benefit agent associated with the second part of an affinity pair from at least one of human hair, human skin, or human nail comprise providing an aqueous solution that is capable of disrupting the affinity pair; and contacting the human hair, skin or nail with the aqueous solution for a time sufficient to disrupt the affinity pair wherein the human hair, skin or nail has been previously contacted with a composition comprising a peptidic component having at least one binding domain which binds to the human hair, skin, or nail with a K_{d} of MB₅₀ value of 10⁻⁵ molar or less and which further comprises the first part of an affinity pair, for a time sufficient for the binding domain to bind to the human hair, skin, or nail; the human hair, skin, or nail having been subsequently contacted with a stable dispersion of particulate benefit agent having average particle size of between 0.01 micron or less to about 75 microns and the second part of the affinity pair.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “g” means the gravitation constant, “rpm” means revolutions per minute,

### General Methods

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents, restriction enzymes and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

### Example 1

### Exemplary Methods for Selection of Peptidic Binding Domains

A suitable library of phage-peptides is generated using the methods described above or the library is purchased from a commercial supplier. After the library of phage-peptides has been generated, they are then contacted with an appropriate amount of the substrate. The library of phage-peptides is dissolved in a suitable solution for contacting the substrate. The test substrate may be suspended in the solution or may be immobilized on a plate or bead. An exemplary solution is a buffered aqueous saline solution containing a surfactant. A suitable solution can be Tris-buffered saline (TBS) with 0.05 to 0.5% TWEEN® 20. The solution may additionally be agitated by any means in order to increase the mass transfer rate of the peptides to the substrate, thereby shortening the time required to attain maximum binding.

Upon contact, a number of the randomly generated phage-peptides will bind to the substrate to form a phage-peptide-substrate complex. Unbound phage-peptide may be removed by washing. After all unbound material is removed, phage-peptides having varying degrees of binding affinities for the substrate may be fractionated by selected washings in buffers having varying stringencies. Increasing the stringency of the buffer used increases the required strength of the bond between the phage-peptide and substrate in the phage-peptide-substrate complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, TWEEN® 20, wherein TWEEN® 20 is exemplary. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is exemplary.

It will be appreciated that phage-peptides having increasing binding affinities for the substrate may be eluted by repeating the selection process using buffers with increasing stringencies. The eluted phage-peptides can be identified and sequenced by any means known in the art.

In one embodiment, the following method for generating of keratin-containing body surface-binding peptides may be used. A library of combinatorially generated phage-peptides is contacted with a substrate (e.g., human hair, skin, or nail) to form phage peptide-substrate complexes. The phage-peptide-substrate complex is separated from uncomplexed peptides and unbound substrate, and the bound phage-peptides from the phage-peptide-substrate complexes are eluted from the complex, for example, by acid treatment. Then, the eluted phage-peptides are identified and sequenced. To identify peptide sequences that bind to the target substrate but not to other substrates (e.g. non-keratin-containing substrates), a subtractive panning step may be added. Specifically, the library of combinatorially generated phage-peptides is first contacted with the non-target to remove phage-peptides that bind to it. Then, the non-binding phage-peptides are contacted with target substrate and the above process is followed. Alternatively, the library of combinatorially generated phage-peptides may be contacted with the non-target and the target simultaneously. Then, the phage-peptide-substrate complexes are separated from the phage-peptide-non-target complexes and the method described above is followed for the desired phage-substrate complexes

Alternatively, a modified phage display screening method for isolating peptides with a higher affinity for one keratin-containing body surface (such as hair) over another keratin-containing surface (such as skin) may be used. In the modified method, the phage-peptide-substrate complexes are formed as described above. Then, these complexes are treated with an elution buffer. Any of the elution buffers described above may be used. In some embodiments, the elution buffer is an acidic solution. Then, the remaining, elution-resistant phage-peptide-substrate complexes are used to directly infect/transfect a bacterial host cell, such as E. coli ER2738. The infected host cells are grown in an appropriate growth medium, such as LB (Luria-Bertani) medium, and this culture is spread onto agar, containing a suitable growth medium, such as LB medium with IPTG (isopropyl β-D-thiogalactopyranoside) and S-Gal™. After growth, the plaques are picked for DNA isolation and sequencing to identify the peptide sequences with a high binding affinity for the substrate of interest. Alternatively, PCR may be used to identify the elution-resistant phage-peptides from the modified phage display screening method, described above, by directly carrying out PCR on the phage-peptide-substrate complexes using the appropriate primers, as described by Janssen et al. in U.S. Patent Application Publication No. 2003/0152976.

### Example 2

### Determination of Peptide Binding Affinities and Specificities

The purpose of this Example was to determine the affinity and specificity of the various hair-binding peptides for hair and pigment surfaces, measured as MB₅₀ values, using an ELISA assay. Each of the short, linear peptides were originally identified using phage display. Unless otherwise indicated, the sequences provided in Example 2 include a C-terminal lysine residue that was biotinylated for detection purposes. Examples of body surface-binding peptide having affinity for various keratin-containing materials (such as hair, skin, and nail) are provided in Table 1, above.

### Synthesis of Hair-Binding Peptides

Hair-binding peptides were synthesized using standard solid phage synthesis method and were biotinylated at the C-terminus lysine residue of binding sequence for detection purposes. The amino acid sequence of the peptides tested are provided in Table 6.

TABLE 6
Hair-binding peptides coupled to biotin via a C-terminal lysine

| Peptide ID | Structure | SEQ ID NO. |
|---|---|---|
| MEA4-biotin | HINKTNPHQGNHHSEKTQRQK-Biotin | 186 |
| Hair2-biotin | AQSQLPDKHSGLHERAPQRYK-Biotin | 187 |
| Hair4-biotin | TPPELAHTPHHLAQTRLTDRK-Biotin | 188 |
| HCP1-biotin | THSTHNHGSPRHTNADAGNPK-Biotin | 189 |

The MB₅₀ measurements of biotinylated peptides binding to hair and to red iron oxide particles (Unipure Red from Sensient Technologies, Milwaukee, Wis.) were made using hair bundles. The hair samples were assembled in bundles consisting of 100 hairs about 1 cm long which were bundled together using narrow tape at one end. The hair bundles were incubated in SUPERBLOCK® blocking buffer (Pierce Chemical Co., Rockford, Ill.) for 1 hour at room temperature (˜22° C.), followed by 3 washes with TBST (TBS in 0.05% TWEEN® 20). Peptide binding buffer consisting of various concentrations of biotinylated peptide in TBST and 1 mg/mL BSA was added to the hair bundles and incubated for 1 hour at room temperature, followed by 6 TBST washes. Then, the streptavidin-horseradish peroxidase (HRP) conjugate (Pierce Chemical Co., Rockford, Ill.) was added to each well (1.0 μg per well), and incubated for 1 h at room temperature, followed by 6 times of washes with TBST. All hair bundles were transferred to new tubes and then the color development and the absorbance measurements were performed following standard protocols. The results were plotted as A₄₅₀ versus the concentration of peptide using GraphPad Prism 4.0 (GraphPad Software, Inc., San Diego, Calif.). The MB₅₀ values were calculated from Scatchard plots and are shown Table 7.

The results, shown in Table 7, demonstrated that the hair binding peptides had better binding affinities for hair than for exemplified particle surface (red iron oxide pigment particles).

TABLE 7
Summary of MB₅₀ Values for Hair-Binding Peptides
Against Hair and Particle Surfaces Peptide ID

| (SEQ ID NO) | Substrate | MB₅₀ (M) |
|---|---|---|
| MEA4 | 90% gray hair | 6.1 × 10⁻⁸ |
| (SEQ ID NO: 76) | Unipure Red particle | 8.7 × 10⁻⁶ |
| Hair2 | 90% gray hair | 2.5 × 10⁻⁷ |
| (SEQ ID NO: 82) | Unipure Red particle | 6.8 × 10⁻⁶ |
| Hair4 | 90% gray hair | 1.5 × 10⁻⁷ |
| (SEQ ID NO: 86) | Unipure Red particle | 5.0 × 10⁻⁶ |
| HCP1 | 90% gray hair | 4.4 × 10⁻⁸ |
| (SEQ ID NO: 110) | Unipure Red particle | 9.8 × 10⁻⁶ |

### Example 3

### Hair Care Peptide 263 (HC263) Mediates Binding of Co-NTA Magnetic Beads to Gray Hair

Approximately 5 mg of 5 mm long hair strands (90% gray, human) were transferred to 2-mL microcentrifuge tubes. Approximately 1 mL of TBST_{0.1} buffer (25 mM Tris, 150 mM NaCl, pH7.2, with 0.1% TWEEN®-20) or the same buffer including varying concentrations of HC263 peptide was added to the hair.

The general design of the hair-binding domain portion of the peptide reagent was generally comprised of a short N-terminus followed by at least 2 hair-binding peptides separated by a peptide linker. The hair-binding domain was then connected, via a peptide spacer (optionally referred to herein as a “peptide bridge”), to the C-terminal polyhistidine region (“his-tag”) having an associative affinity for metal chelate resins (e.g., cobalt-NTA resins). Sequences of the peptides used to assemble the peptide-based reagents are shown in Table 8.

TABLE 8
Peptides used prepare the peptide-based reagents in Examples 3 and 4.

| Peptide ID | Sequence | SEQ ID NO: |
|---|---|---|
| Hair2 | AQSQLPDKHSGLHERAPQRY | 82 |
| Gray3 | HDHKNQKETHQRHAA | 120 |
| Hair2Rnd1 | DRSKLYQSLEHRQPPGAHAQ | 190 |
| Gray3Rnd4 | AHDAKHEHRKQNHTQ | 191 |
| F4Rnd | QTHVSQPFLFHD | 192 |
| IB5A | TPPELLHGAPRS | 89 |
| Rfe1 | WAPEKDHMQLMK | 193 |
| CA3 | NGNNHTDIPNRSSYTGGSFA | 194 |
| CA4 | SDETGPQIPHRRPTW | 195 |

The mixtures were shaken on a Nutator for one hour at room temperature (˜22° C.). The hairs were washed three times with TBST_{0.5} buffer (25 mM Tris, 150 mM NaCl, pH 7.2, with 0.5% TWEEN®-20). After washing, the hairs were resuspended in 900 μL Talon buffer (50 mM sodium phosphate, pH 8.0, 300 mM sodium chloride, 0.01% TWEEN®-20). A subset of hair/peptide conjugates were washed additionally one time with 1 mL of 0.25% SLES (Sodium Lauryl Ether Sulfate) for 5 min at room temperature on the Nutator, then washed two times with 1 mL TBST_{0.1} buffer. Approximately 0.2 mg of TALON™ beads (DYNABEADS® TALONT™, Invitrogen, Catalog#101.01D) were washed twice with TALON™ buffer on the DYNAL® MPC™ magnet and added to each reaction in 100 μL TALON™ buffer. The magnetic beads were coated with Co-NTA, which specifically binds to the six histidine residues incorporated into the peptide (C-terminal his tag). The hair and beads were incubated for 10 minutes with gentle shaking before the tubes were placed on the magnet.

Beads binding to the hair were expected to co-migrate on the sides of the tubes once applied to the magnet. The strength of binding was visually assessed and rated on a scale of 0 (little or no observed binding) to 5 (highest amount of observed binding). Table 9 summarizes the results, which show optimal binding at a concentration of 0.2 μM and 0.02 μM of HC263 (SEQ ID NO: 196). At 0.007 μM a decrease of binding was observed. Binding at 0.2 μM peptide was not compromised by washing the hair with SLES.

TABLE 9
Peptide mediated binding of magnetic beads to hair.

| Peptide | Conc. | SLES Present? | Strength of binding |
|---|---|---|---|
| ID | (μM) | (Yes/No) | (5 highest, 0 lowest) |
| HC263 | 0.2 | No | 5 |
| HC263 | 0.2 | Yes | 5 |
| HC263 | 0.02 | No | 5 |
| HC263 | 0.007 | No | 2 |
| HC263 | 0.002 | No | 2 |
| No | 0 | No | 0 |
| peptide | | | |
| (control) | | | |

### Example 4

### Peptide-Mediated Binding of Co-NTA Magnetic Beads to Gray Hair

To determine the requirements of the peptide to achieve the effect observed in Example 3, additional peptides were tested (Table 10). Engineered peptides HC263, HC264, HC204, HC205, and HC214, which are each an exemplary embodiment of the invention, each have polyhistidine tags that allow binding to the Co-NTA coated magnetic beads. HC352, HC423, and HC424 have two or more hair-binding domains like HC263, but do not have a polyhistidine tag and were used as controls. HC264 has the identical sequence composition as HC263, except for randomized hair binding sequences.

TABLE 10
Peptides tested to determine required domains for peptide mediated binding of
magnetic beads to hair.

| | | Binding | Part of |
|---|---|---|---|
| | Description | Domain | affinity |
| Peptide | (SEQ ID NO.) | Hair | pair |
| HC263 | MASGSDPS-Hair2- | HP2-Gray3 | HHHHHH |
| | GPEPEPEPEPIPEPPKEAPVVIEKPKPKPKPKPKPPA- | | (SEQ ID |
| | Gray3-GSGGGGSP-HHHHHH | | NO: 235) |
| | (SEQ ID NO: 196) | | |
| HC264 | MASGSDPS-Hair2Rnd1- | HP2Randomized- | HHHHHH |
| | GPEPEPEPEPIPEPPKEAPVVIEKPKPKPKPKPKPPA- | Gray3Randomized | (SEQ ID |
| | Gray3Rnd4-GSGGGGSP-HHHHHH | | NO: 235) |
| | (SEQ ID NO: 197) | | |
| HC214 | GSDPG-Gray3-GGG-Gray3-GGGGSGGGGSG- | Gray3- | HHHHHH |
| | HHHHHH | Gray3 | (SEQ ID |
| | (SEQ ID NO: 198) | | NO: 235) |
| HC204 | GSDPG-F4Rnd-GGG-F4Rnd-GGGGSGGGGSG- | F4Rnd- | HHHHHH |
| | HHHHHH | F4Rnd | (SEQ ID |
| | (SEQ ID NO: 199) | | NO: 235) |
| HC205 | GSDPG-IB5A-GGGGSGGGGSG-HHHHHH | IB5A | HHHHHH |
| | (SEQ ID NO: 200) | | (SEQ ID |
| | | | NO: 235) |
| HC352 | MASGSDPS-Hair2- | HP2-Gray3 | none |
| (control) | GPEPEPEPEPIPEPPKEAPVVIEKPKPKPKPKPKPPA- | | |
| | Gray3-GSGGGGSP-Rfe1-GGAGGAG-Rfe1-GK | | |
| | (SEQ ID NO: 201) | | |
| HC423 | MASGSDPS-Hair2- | HP2-Gray3 | none |
| (control) | GPEPEPEPEPIPEPPKEAPVVIEKPKPKPKPKPKPPA- | | |
| | Gray3-GPGGGSGGGSGGGSGGGSPA-CA3- | | |
| | GGAGGAG-CA3-GK | | |
| | (SEQ ID NO: 202) | | |
| HC424 | MASGSDPS-Hair2- | HP2-Gray3 | none |
| (control) | GPEPEPEPEPIPEPPKEAPVVIEKPKPKPKPKPKPPA- | | |
| | Gray3-GPGGGSGGGSGGGSGGGSPA-CA4- | | |
| | GGAGGAG-CA4-GK | | |
| | (SEQ ID NO: 203) | | |

Bold = binding unit within formula listed by name.

The hair binding assay of Example 3 was used to assess the strength of binding of the test peptides in Table 10. Results are summarized in Table 11.

TABLE 11
Peptide-mediated binding of magnetic beads to hair.

| Peptide | Concentration | Strength of binding |
|---|---|---|
| ID | (μM) | (5 highest, 0 lowest) |
| HC205 | 0.2 | 0 |
| HC206 | 0.2 | 0 |
| HC214 | 0.2 | 0 |
| HC352 | 0.2 | 0 |
| HC352 | 0.02 | 0 |
| HC423 | 0.2 | 3 |
| HC423 | 0.02 | 0 |
| HC424 | 0.2 | 0 |
| HC424 | 0.02 | 0 |
| HC263 | 0.2 | 5 |
| HC263 | 0.02 | 5 |
| HC263 | 0.002 | 2 |
| HC264 | 0.2 | 5 |
| HC264 | 0.02 | 5 |
| HC264 | 0.002 | 2 |
| None | 0 | 0 |
| (control) | | |

### Example 5

### HC263-Mediated Binding of Magnetic Beads to Hair

Peptide HC263 (SEQ ID NO: 196) was tested for peptide-mediated binding of magnetic beads to various surfaces: human hair, yak hair, wool, cotton, SONTARA® (a nonwoven, spunlaced textile sheet fabric, E.I. duPont de Nemours and Company, Inc., Wilmington, Del.), and cellulose (filter paper). Approximately 5 mg of human hair (90% gray), yak hair, wool, cotton, SONTARA®, or filter paper were added to 2-mL microcentrifuge tubes. Approximately 1 mL of TBST_{0.1} buffer (25 mM Tris, 150 mM NaCl, pH 7.2, with 0.1% TWEEN®-20) containing 0.2 μM HC263 was added. The mixtures were shaken on a Nutator (BD Diagnostics, Franklin Lakes, N.J.) for 30 minutes at room temperature (˜22° C.). The samples were washed three times with TBST_{0.5} buffer (25 mM Tris, 150 mM NaCl, pH 7.2, with 0.5% TWEEN®-20). After washing, the samples were re-suspended in 900 μL TALON™ (Clontech, Mountain View, Calif.) buffer (50 mM sodium phosphate, pH 8.0, 300 mM sodium chloride, 0.01% TWEEN®-20). Approximately 0.2 mg of TALON™ beads (DYNABEADS® TALON™, Invitrogen, Carlsbad, Calif.; Catalog#101.01D) were washed twice with TALON™ buffer on the DYNAL® MPC™ magnet (Invitrogen) and added to each reaction in 100-μL TALON™ buffer.

The strength of binding of the magnetic beads to the various surfaces was evaluated by visualization of scanning electron microscope (SEM) images taken at 1000-fold magnification. A relative scoring system was used to rank the binding strength of the beads to various surfaces based on the SEM images (0=little or no binding, 5=strong binding). Table 12 shows that strong binding of magnetic beads mediated by peptide HC263 only occurs with human hair.

TABLE 12
Peptide-mediated binding of magnetic beads to various surfaces.

| | Strength of binding |
|---|---|
| Surface | (5 highest, 0 lowest) |
| Human hair | 5 |
| Yak hair | 1 |
| wool | 0 |
| cotton | 0 |
| SONTARA ® | 0 |
| cellulose | 0 |

### Example 6

### Deposition Utilizing Biotin-Peptide and Streptavidin-Polystyrene Particles

A biotinylated peptide and streptavidin-coated bead system was used to illustrate the coupling of beads to hair in accordance with the methods and compositions of this invention. Sequential deposition was achieved by the following process:

A human hair sample was placed in an aqueous solution containing 20 mM of HCP5 dimer peptide (SEQ ID NO: 204) derived in accordance with the methods set forth herein. The HCP5 dimer comprises two HCP5 hair-binding peptides (SEQ ID NO: 112) originally derived from a phage display library coupled together using short spacer (HCP5-GGSGPGSGG-HCP5). The HCP5 dimer was purchased from American Peptide Co., Inc. (Sunnyvale, Calif.) in both biotinylated (SEQ ID NO: 205) and non-biotinylated (SEQ ID NO: 204) versions (a single lysine residue was added to the C-terminus of the biotinylated version to facilitate biotin coupling).

Hair samples were pretreated by exposing the hair samples to either the biotinylated or the non-biotinylated peptide. Ten strands of natural dark brown hair (International Hair Importers), three inches in length, were pre-washed with 2% SLES for 30 seconds, then rinsed extensively with DI water. The hair strands were then immersed in 10 mL of a 5-20 micromolar solution of peptide for up to one hour. Ten hair samples were exposed to the biotinylated peptide and ten hair samples were exposed to non-biotinylated peptide. Peptide solutions were prepared in either DI water or a Tris-HCl buffer at pH 7.2 (ionic strength ranging from 5 mM-150 mM) for up to 1 hour. The peptide treatment step is expected to result in deposition of the peptide onto the hair sample. Peptide treated hair samples were subsequently rinsed by immersion into a solution of the peptide treatment buffer for 30 seconds to remove excess peptide. Next, the peptide-treated hair samples were incubated with 40 nm fluorescently-labeled streptavidin-coated polystyrene particles (Invitrogen, Carlsbad, Calif.) to allow the binding between biotin and streptavidin. Particle dispersions were created in various media including DI water and Tris Buffered Saline buffer at pH 7.2 (ionic strength ranging from 5 mM-150 mM) containing up to 0.1% TWEEN. Binding was permitted to take place during an incubation period for up to 24 hours at room temperature (˜22° C.) in the dark. Samples were continuously inverted end-over-end during the incubation period.

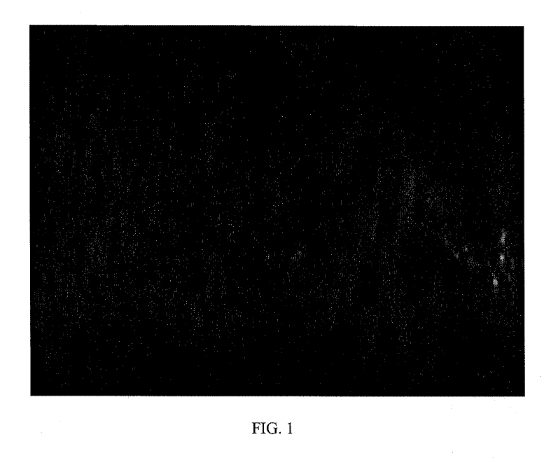

After the incubation period ended, fluorescence microscopy was used to view the binding of 40 nm fluorescently-labeled streptavidin-coated polystyrene particles to hair. FIG. 1 illustrates that the biotinylated HCP5 dimer peptide bound the particles to hair, as evidenced by the observed fluorescence.

### Example 7

### Deposition Utilizing Biotin-Peptide and Streptavidin Iron Oxide

A biotinylated peptide and streptavidin-bead system was used to color hair. Sequential deposition was achieved by the following process.

Miniature hair tresses of approximately 1 inch in length containing 100% unpigmented hair were placed in an aqueous solution with 20 mM of peptides (in DI water or Tris Buffered Saline, pH 7.2, 5-150 mM ionic strength), for up to one hour at room temperature, derived in accordance with the methods set forth herein. Peptide HC260 was recombinantly produced in E. coli. Methods to recombinantly produce and isolate peptide reagents using an E. coli production host are well in the art (for example, Examples 17-20 of U.S. Pat. No. 7,285,264; incorporated herein by reference. The sequences of the functional peptide units use to create the HCP5 dimer (“(HCP5)2”), HC260 are provided in Table 13. The formula of the peptide reagents (i.e., engineered peptides) are provided in Table 14. HC260 was engineered to contain a streptavidin-binding peptide tag (SB33N).

TABLE 13
Peptide units used prepare the peptide-based reagents in Table 14.

| Peptide ID | Sequence | SEQ ID NO: |
|---|---|---|
| HCP5 | HHGTHHNATKQKNHV | 112 |
| Hair2 | AQSQLPDKHSGLHERAPQRY | 82 |
| Gray3 | HDHKNQKETHQRHAA | 120 |
| SB33N | MLSENWLTNHPQN | 206 |

TABLE 14
Peptide reagents tested to determine required domains for
peptide mediated binding of magnetic beads to hair.

| | Description of Constructs | |
|---|---|---|
| Peptide | (Formula) | SEQ ID NO: |
| HCP5-dimer | HCP5-GGSGPGSGG-HCP5 | 204 |
| “(HCP5)2” | | |
| HC260 | MASGSDPS-Hair2- | 207 |
| | GPEPEPEPEPIPEPPKEAPVVIEKPKPKP | |
| | KPKPKPPA-Gray3-GSGGGGSP-SB33N- | |
| | GGAGGAG-SB33N-GK | |

Bold = binding unit within formula listed by name.

Hair samples were pretreated by exposing the hair to different peptides. The peptide created a layer on the hair. Hair samples with a peptide layer were rinsed with SLES solution to remove excess peptide. Next, the peptide-treated hair samples were incubated with streptavidin tagged iron oxide particles (Bangs Laboratories, Inc., Fishers, Ind.; Ademtech, Inc., Pessas, France) to allow the binding between biotin and streptavidin or between the streptavidin-binding domain in HC260 and streptavidin. Particles were purchased in dispersed form and were diluted to 0.025-1 wt % by adding a specified amount of either DI water or Coupling Buffer from Ademtech. Binding was permitted to take place during an incubation period for up to 24 hours at room temperature (˜22° C.) in the dark. Samples were continuously inverted end-over-end during the binding duration incubation period.

Table 15 shows that colored streptavidin-coated iron oxide beads (500 nm) deposited on hair samples with biotinylated peptide (HCP5)2, whereas colored streptavidin-coated iron oxide beads (500 nm) did not deposit as well on hair samples with non-biotinylated peptide (HCP5)2. Hair sample #3 was coated with non-biotinylated (HCP5)2 peptide, and hair sample #4 was coated with biotinylated (HCP5)2 peptide. A color scale for the associated color intensity of iron oxide treated hair was defined as (0-5), where 0 is no color deposition and 5 is the deepest color. In Table 15, hair sample #3 has an associated color intensity value of 0, while hair sample #4 has an associated color intensity value of 4.

TABLE 15
Visual color assessment of hair samples treated with
streptavidin-coated iron oxide beads and HCP5-dimer peptide
(HCP5)2 comparing biotinylated and non-biotinylated versions.

| Hair Sample number | Samples treatment | Color scale |
|---|---|---|
| 3 | streptavidin-coated | 0 |
| | beads deposited with | |
| | non-biotinylated | |
| | (HCP5)2 peptide | |
| 4 | streptavidin-coated | 4 |
| | beads deposited with | |
| | biotinylated peptide | |
| | (HCP5)2 | |

Table 16 shows the visual color assessment of hair samples treated with various peptide constructs and streptavidin-coated iron oxide beads using a sequential treatment method. Hair samples treated with peptide constructs containing a streptavidin-binding partner (i.e. either biotin or a streptavidin-binding peptide motif) enhanced hair coloration when treated with colored streptavidin-coated iron oxide beads.

The streptavidin binding domains is HC260 was SB33N which have estimated streptavidin-binding K_{d} values of 10⁻⁴ M. Table 16 shows the treated tresses of hair. On the color scale, the peptides were scored as follows: (HCP5)2=0, (HCP5) 2-biotin=4, HC260=2.

TABLE 16
Visual color assessment of hair samples coated with various
biotinylated peptides and subsequently streptavidin coated
beads (sequential coloring method).

| | Reported Binding | |
|---|---|---|
| | constant of streptavidin | |
| | binding partner in | |
| Samples treatment | construct (M) | Color scale |
| (HCP5)2 | | 0 |
| (HCP5) 2-biotin | 10⁻¹⁵ | 4 |
| HC260 | 10⁻⁴ | 2 |

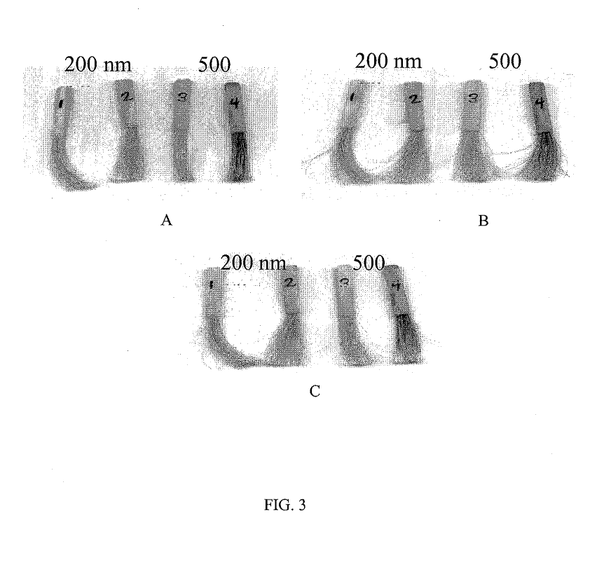

Tables 17-19 demonstrate visual color assessment of hair coloration using streptavidin-coated iron oxide particles. Samples #3 and #4 correspond to non-biotinylated and biotinylated versions of the HCP5-dimer [(HCP5)2], respectively, with 500 nm particles and samples #1 and #2 correspond to peptides (HCP5)2 and (HCP5)2—biotin respectively with 200 nm particles respectively. Furthermore, water wash and 0.25% SLES wash steps are shown. Using the previously-described color scale, in the initial data, subsequent to exposure to the coated peptide, but before washing with water, the color measurements were as follows: sample 1=0, sample 2=0, sample 3=0, and sample 4=4 (Table 17; FIG. 3A). After washing with water, the color measurements were as follows: sample 1=0, sample 2=0, sample 3=0, and sample 4=3 (Table 18; FIG. 3B). In the 0.25% SLES (sodium lauryl ether sulfate) wash data: sample 1=0, sample 2=0, sample 3=0, and sample 4=2 (Table 19; FIG. 3C).

TABLE 17
Visual color assessment of hair coloration using 200 nm or 500 nm
streptavidin-coated iron oxide particles (initial conditions)

| | Bead diameter | | |
|---|---|---|---|
| Sample # | (nm) | Wash Conditions | Color scale |
| 1 | 200 | initial | 0 |
| (HCP5)2 | | | |
| 2 | 200 | initial | 0 |
| (HCP5)2-biotin | | | |
| 3 | 500 | initial | 0 |
| (HCP5)2 | | | |
| 4 | 500 | initial | 4 |
| (HCP5)2-biotin | | | |

TABLE 18
Visual color assessment of hair coloration using 200 nm or 500 nm
streptavidin-coated iron oxide particles (after water wash)

| | Bead diameter | | |
|---|---|---|---|
| Sample # | (nm) | Wash Conditions | Color scale |
| 1 | 200 | water wash | 0 |
| (HCP5)2 | | | |
| 2 | 200 | water wash | 0 |
| (HCP5)2-biotin | | | |
| 3 | 500 | water wash | 0 |
| (HCP5)2 | | | |
| 4 | 500 | water wash | 4 |
| (HCP5)2-biotin | | | |

TABLE 19
Visual color assessment of hair coloration using 200 nm or 500 nm
streptavidin-coated iron oxide particles (after 0.25% SLES wash)

| | Bead diameter | | |
|---|---|---|---|
| Sample # | (nm) | Wash Conditions | Color scale |
| 1 | 200 | 0.25% SLES Wash | 0 |
| (HCP5)2 | | | |
| 2 | 200 | 0.25% SLES Wash | 0 |
| (HCP5)2-biotin | | | |
| 3 | 500 | 0.25% SLES Wash | 0 |
| (HCP5)2 | | | |
| 4 | 500 | 0.25% SLES Wash | 2 |
| (HCP5)2-biotin | | | |

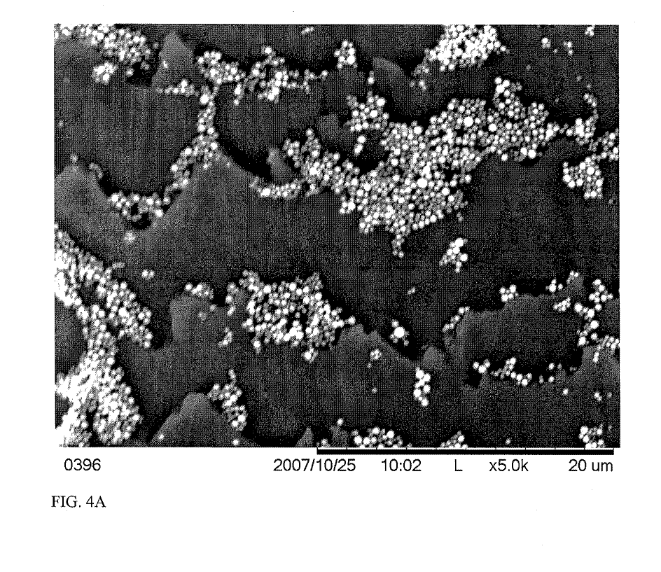

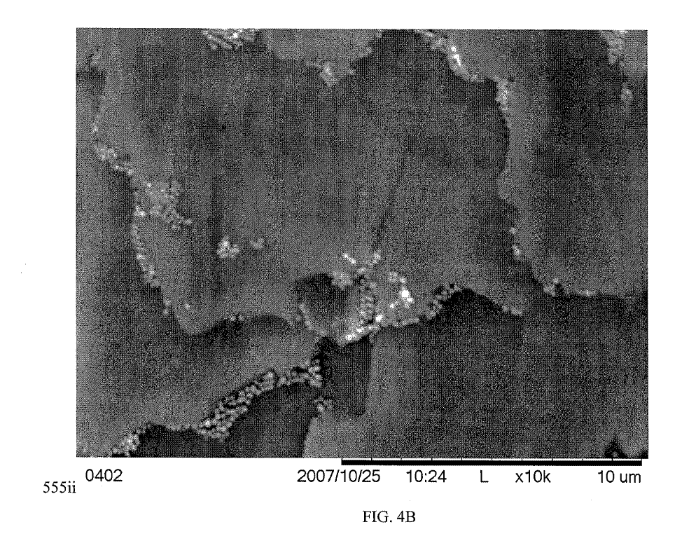

Example 7 demonstrates the effect of particle size on the human perception of color. Humans can perceive color that is in about the 400 nm to 700 nm range. Ultraviolet light, i.e., 200-400 nm is not visible to humans. As demonstrated in Tables 17-19, only the (HCP5)2-biotin-treated hair treated with 500 nm beads exhibited perceptible color. The (HCP5)2-biotin-treated hair treated with 200 nm beads did not exhibited perceptible color, even though 200 nm beads were deposited on the hair. See FIGS. 4A and 4B.

### Example 8

### Hair Coloring with Sequentially Applied Peptide and Pigments

Minitresses of natural white hair (0.5-1 cm wide, 1 inch long) were obtained from International Hair Inc., Florence, S.C. The hair tresses were hand washed with 2% sodium lauryl ether sulfate (SLES, Rhodapex ES-2K) solution, followed by DI water rinse and air dry before testing. Peptides HC634, HC635, HC636, HC637, HC638, HC639, HC640, HC641, HC642, HC643, HC644 and HC645 were each weighed and dissolved into pH 7.5, 25 mM Tris buffer at concentration 0.7 mg/mL. The hair-binding domain comprises hair-binding peptides HP2E (SEQ ID NO: 84) and Gray3A (SEQ ID NO: 77) joined by a tonB linker (SEQ ID NO: 208). The binding domain was coupled via a bridge peptide (SEQ ID NO: 209) to various peptides (i.e., the first part of the affinity pair) having an affinity for the pigment (i.e., silica coated red iron oxide).

TABLE 20
Hair coloring peptide conjugates with different pigment-binding hands

| | Binding Domain | | Bridge | | First part of Affinity Pair | |
|---|---|---|---|---|---|---|
| Peptide ID | Formula of | Chrg¹ at | Bridge | Chrg¹ at | | Chrg¹ at |
| (SEQ ID NO:) | Hair binding domain | pH 7.5 | (SEQ ID NO: 209) | pH 7.5 | | pH 7.5 |
| HC634 | HP2E-TonB-Gray3A | 3.4 | (GSGGGGSP)₂ | 0 | (KQPN)₅ | 5 |
| (SEQ ID NO: 211) | | | | | (SEQ ID NO: 223) | |
| HC635 | HP2E-TonB-Gray3A | 3.4 | (GSGGGGSP)₂ | 0 | (KQPN)₉ | 9 |
| (SEQ ID NO: 212) | | | | | (SEQ ID NO: 224) | |
| HC636 | HP2E-TonB-Gray3A | 3.4 | (GSGGGGSP)₂ | 0 | (KQPN)₁₃ | 13 |
| (SEQ ID NO: 213) | | | | | (SEQ ID NO: 225) | |
| HC637 | HP2E-TonB-Gray3A | 3.4 | (GSGGGGSP)₂ | 0 | (KQGPQ)₉ | 9 |
| (SEQ ID NO: 214) | | | | | (SEQ ID NO: 226) | |
| HC638 | HP2E-TonB-Gray3A | 3.4 | (GSGGGGSP)₂ | 0 | (KQGPQG)₉ | 9 |
| (SEQ ID NO: 215) | | | | | (SEQ ID NO: 227) | |
| HC639 | HP2E-TonB-Gray3A | 3.4 | (GSGGGGSP)₂ | 0 | (KGQGPQG)₉ | 9 |
| (SEQ ID NO: 216) | | | | | (SEQ ID NO: 228) | |
| HC640 | HP2E-TonB-Gray3A | 3.4 | (GSGGGGSP)₂ | 0 | (KQPNHQPQ)₅ | 7.5 |
| (SEQ ID NO: 217) | | | | | (SEQ ID NO: 229) | |
| HC641 | HP2E-TonB-Gray3A | 3.4 | (GSGGGGSP)₂ | 0 | (HQPNHQPQ)₅ | 5 |
| (SEQ ID NO: 218) | | | | | (SEQ ID NO: 230) | |
| HC642 | HP2E-TonB-Gray3A | 3.4 | (GSGGGGSP)₂ | 0 | (HSPNHSPS)₅ | 5 |
| (SEQ ID NO: 219) | | | | | (SEQ ID NO: 231) | |
| HC643 | HP2E-TonB-Gray3A | 3.4 | (GSGGGGSP)₂ | 0 | HTSPQHHTSPHPHKNPQ | 4 |
| (SEQ ID NO: 220) | | | | | HTSPTPN | |
| | | | | | (SEQ ID NO: 232) | |
| HC644 | HP2E-TonB-Gray3A | 3.4 | (GSGGGGSP)₂ | 0 | HTKQNHTSPTPHHTNSP | 3.5 |
| (SEQ ID NO: 221) | | | | | QHTSPHP | |
| | | | | | (SEQ ID NO: 233) | |
| HC645 | HP2E-TonB-Gray3A | 3.4 | (GSGGGGSP)₂ | 0 | HNNRQHTSPHPQHTKQ | 3.5 |
| (SEQ ID NO: 222) | | | | | NHTSPTPH | |
| | | | | | (SEQ ID NO: 234) | |

¹= net charge of respective portion of the peptide reagent.

Each hair tress was incubated with 0.6 mL peptide solution in 1.7-mL microcentrifuge vials on a rotator for 15 minutes. Duplicates of hair tresses were run for each peptide solution. Then the tresses were taken out of vials and rinsed with DI water, blotted with paper towels and placed into a new-labeled vial for pigment application. Silica-coated iron oxide particles were made according to the methods disclosed in U.S. Pat. No. 2,885,366, incorporated herein. Silica-coated red ion oxide dispersions were prepared with an isoelectric point of 2 (IEP2) and 5 (IEP5). Approximately 0.6 mL of 1% pigment dispersion in pH 7.5, 25 mM Tris buffer was added to each vial and incubated with peptide treated hair tresses on rotator for 10 minutes. Afterwards, the hair tresses were taken out and rinsed with tap water, paper towel blot and dry with air. L*a*b* score was taken for each hair tress using X-Rite spectrophotometer (X-Rite Incorporated, Grand Rapids, Mich.) on four different spots on both sides of hair samples, and average of L*a*b* score was used for color uptake deltaE calculation (ΔE), for which untreated natural white hair was used as reference. The no peptide controls were tested by applying the hair tresses to the pigment dispersion directly with the same procedure.

Delta E values can be calculated using equation (1) below:

Delta E=((L*₁−L*₂)²+(a*₁−a*₂)²+(b*₁−b*₂)²)^{1/2} (1)

where L*=the lightness variable and a* and b* are the chromaticity coordinates of CIELAB colorspace as defined by the International Commission of Illumination (CIE) (Minolta, Precise Color Communication—Color Control From Feeling to Instrumentation, Minolta Camera Co., 1996).

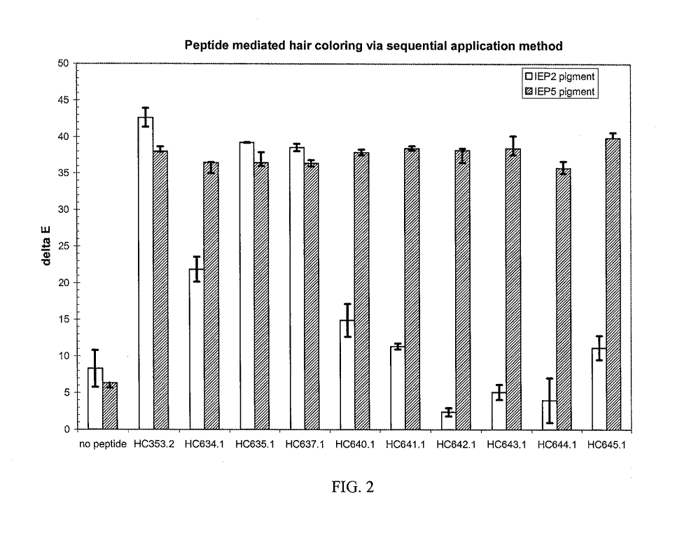

Peptide mediated hair coloring results are summarized in FIG. 2. Without peptide, both IEP2 and IEP5 silica-coated iron oxide pigments produced about 5-8 deltaE (ΔE) units background color uptake, while peptide mediated pigment deposition produced 2 to 43 deltaE (ΔE) units color uptake with IEP2 pigment, and 37-42 deltaE units color uptake with IEP5 pigment. The peptide-pigment systems were demonstrated to color hair effectively.

### Example 9

### Removal of Benefit Agent from Peptidic Component

Step 1: Hair tresses will be incubated for about 15 min with a peptide solution containing a peptide component of the invention, for example, (HCP5)2-biotin, and will be rinsed with water and blotted. The incubated hair tresses will then be treated for about 10 min with a stable dispersion of particulate benefit agent of the invention. For example, the incubated hair tresses will be treated with a stable dispersion comprising streptavidin-coated iron oxide beads, then will be rinsed with water and blotted.

Step 2: Hair tresses that are colored according to the methods of the invention can be treated to remove the particulate benefit agent of the invention. For example, if the benefit agent is a colorant, the colorant can be removed. The hair tress can be prepared according to step 1. The hair tress will be treated with an aqueous solution, for example, 0.25% SLES wash, 1 or more times to disrupt the affinity of biotin and streptavidin and release the streptavidin-coated iron oxide beads from the hair tresses.

What is claimed:

1. A cosmetic system comprising:
a peptidic component comprising at least one binding domain which binds to at least one of human hair, human skin or human nail with a K_{d} or MB₅₀ value of 10⁻⁵ molar or less and which further comprises the first part of an affinity pair; and
a stable dispersion of particulate benefit agent having average particle size of between about 0.01 micron and about 75 microns and the second part of the affinity pair;
the at least one binding domain has a greater binding affinity for the human hair, skin or nail than it has for the particles of the dispersion.

2. The cosmetic system of claim 1 wherein the average particle size of between about 0.01 micron and about 75 microns is measured by a method selected from the group consisting of dynamic light scattering and laser diffraction.

3. The cosmetic system of claim 1 wherein the binding domain comprises a plurality of sub-domains.

4. The cosmetic system of claim 1 wherein the association between the benefit agent and second part of the affinity pair is non-covalent.

5. The cosmetic system of claim 1, wherein the binding domain further preferentially binds to human hair, skin or nail over wool, cashmere, or yak hair.

6. The cosmetic system of claim 1 wherein the binding domain further preferentially binds to human hair, skin or nail over cotton or modified cellulosic fiber.

7. The cosmetic system of claim 1, wherein the binding domain further preferentially binds to human hair, skin or nail over metal, ceramic, porcelain, glass, silk, wood, polyester, or polyvinylchloride.

8. The cosmetic system of claim 1, wherein the particulates are nanoparticles.

9. The cosmetic system of claim 1, wherein the affinity pair is ionic bond-based, hydrogen bond-based, hydrophobic bond-based, chelation-based, biological affinity-based, or the affinity pair is based on a combination thereof.

10. The cosmetic system of claim 1, wherein the affinity pair is based on the affinity of biotin to avidin, biotin to streptavidin, streptavidin tags to streptavidin, maltose binding protein to maltose, maltose binding protein to amylase, polyhistidine tag to a metal, glutathione S-transferase to glutathione, epitope tag to an antibody, or a combination thereof.

11. The cosmetic system of claim 10, wherein the metal is a divalent metal.

12. The cosmetic system of claim 11, wherein the metal is cobalt, copper, nickel, zinc, or a combination thereof.

13. The cosmetic system of claim 10, wherein the epitope tag is HA-tag, FLAG-tag, E-tag, S-tag, or myc-tag.

14. The cosmetic system of claim 1, wherein the benefit agent is a sunscreen agent, conditioning agent, encapsulated fragrance, antimicrobial, antidandruff, antifungal, odor control agent, encapsulated bioactive agent, hair removal agent, antiacne agent, or coloring agent.

15. The cosmetic system of claim 14, wherein the coloring agent is a colored pigment, colored particle, or a combination thereof.

16. The cosmetic system of claim 1, wherein the binding domain has a 2-fold greater binding affinity for the human hair, skin or nail than it has for the particles of the dispersion.

17. The cosmetic system of claim 1, wherein the stable dispersion is charge stabilized.

18. The cosmetic system of claim 1 wherein the absolute value of the zeta potential of the particulate benefit component is at least 25 mV.

19. The cosmetic system of claim 1, wherein the stable dispersion is sterically stabilized.

20. The cosmetic system of claim 1, wherein the stable dispersion comprises a dispersant.

21. The cosmetic system of claim 20, wherein the dispersant is an ionic dispersant or non-ionic dispersant.

22. The cosmetic system of claim 1, wherein the first binding domain is identified using biopanning.

23. The cosmetic system of claim 1, wherein the first binding domain is identified using phage display, bacterial display, yeast display, ribosome display, mRNA display, or a combination thereof.

24. The cosmetic system of claim 1, wherein the binding domain has a binding affinity affinity for human hair, human skin or human nail, as measured by a K_{d} or MB₅₀ value of about 10⁻⁵ molar to about 10⁻¹⁰ molar.

25. A method of applying a benefit agent to at least one of human hair, human skin, or human nail comprising:
contacting the human hair, skin, or nail with a composition comprising a peptidic component having at least one binding domain which binds to at least one of the human hair, skin, or nail with a K_{d} or MB₅₀ value of 10⁻⁵ molar or less, and which further comprises the first part of an affinity pair, for a time sufficient for the binding domain to bind to the human hair, skin, or nail; and
subsequently applying to the human hair, skin, or nail a stable dispersion of particulate benefit agent having average particle size of between about 0.01 micron to about 75 microns and the second part of the affinity pair;
wherein the at least one binding domain has greater binding affinity for the human hair, skin, or nail than it has for the particles of the dispersion.

26. The method of claim 25 further comprising contacting the human hair, skin, or nail with an aqueous solution before subsequently contacting the human hair, skin, or nail with the stable particulate dispersion.


SEQUENCE LISTING (235 sequences)

SEQ ID NO: 1 - protein, 12 aa, Artificial Sequence
  Synthetic construct
RVPNKTVTVDGA

SEQ ID NO: 2 - protein, 12 aa, Artificial Sequence
  Synthetic construct
DRHKSKYSSTKS

SEQ ID NO: 3 - protein, 12 aa, Artificial Sequence
  Synthetic construct
KNFPQQKEFPLS

SEQ ID NO: 4 - protein, 12 aa, Artificial Sequence
  Synthetic construct
QRNSPPAMSRRD

SEQ ID NO: 5 - protein, 12 aa, Artificial Sequence
  Synthetic construct
TRKPNMPHGQYL

SEQ ID NO: 6 - protein, 12 aa, Artificial Sequence
  Synthetic construct
KPPHLAKLPFTT

SEQ ID NO: 7 - protein, 12 aa, Artificial Sequence
  Synthetic construct
NKRPPTSHRIHA

SEQ ID NO: 8 - protein, 12 aa, Artificial Sequence
  Synthetic construct
NLPRYQPPCKPL

SEQ ID NO: 9 - protein, 12 aa, Artificial Sequence
  Synthetic construct
RPPWKKPIPPSE

SEQ ID NO: 10 - protein, 12 aa, Artificial Sequence
  Synthetic construct
RQRPKDHFFSRP

SEQ ID NO: 11 - protein, 12 aa, Artificial Sequence
  Synthetic construct
SVPNKXVTVDGX

SEQ ID NO: 12 - protein, 12 aa, Artificial Sequence
  Synthetic construct
TTKWRHRAPVSP

SEQ ID NO: 13 - protein, 12 aa, Artificial Sequence
  Synthetic construct
WLGKNRIKPRAS

SEQ ID NO: 14 - protein, 12 aa, Artificial Sequence
  Synthetic construct
SNFKTPLPLTQS

SEQ ID NO: 15 - protein, 12 aa, Artificial Sequence
  Synthetic construct
SVSVGMKPSPRP

SEQ ID NO: 16 - protein, 7 aa, Artificial Sequence
  Synthetic construct
DLHTVYH

SEQ ID NO: 17 - protein, 7 aa, Artificial Sequence
  Synthetic construct
HIKPPTR

SEQ ID NO: 18 - protein, 7 aa, Artificial Sequence
  Synthetic construct
HPVWPAI

SEQ ID NO: 19 - protein, 7 aa, Artificial Sequence
  Synthetic construct
MPLYYLQ

SEQ ID NO: 20 - protein, 26 aa, Artificial Sequence
  Synthetic construct
HLTVPWRGGGSAVPFYSHSQITLPNH

SEQ ID NO: 21 - protein, 41 aa, Artificial Sequence
  Synthetic construct
GPHDTSSGGVRPNLHHTSKKEKRENRKVPFYSHSVTSRGNV

SEQ ID NO: 22 - protein, 7 aa, Artificial Sequence
  Synthetic construct
KHPTYRQ

SEQ ID NO: 23 - protein, 7 aa, Artificial Sequence
  Synthetic construct
HPMSAPR

SEQ ID NO: 24 - protein, 7 aa, Artificial Sequence
  Synthetic construct
MPKYYLQ

SEQ ID NO: 25 - protein, 7 aa, Artificial Sequence
  Synthetic construct
MHAHSIA

SEQ ID NO: 26 - protein, 12 aa, Artificial Sequence
  Synthetic construct
AKPISQHLQRGS

SEQ ID NO: 27 - protein, 12 aa, Artificial Sequence
  Synthetic construct
APPTPAAASATT

SEQ ID NO: 28 - protein, 12 aa, Artificial Sequence
  Synthetic construct
DPTEGARRTIMT

SEQ ID NO: 29 - protein, 12 aa, Artificial Sequence
  Synthetic construct
LDTSFPPVPFHA

SEQ ID NO: 30 - protein, 12 aa, Artificial Sequence
  Synthetic construct
LDTSFHQVPFHQ

SEQ ID NO: 31 - protein, 11 aa, Artificial Sequence
  Synthetic construct
LPRIANTWSPS

SEQ ID NO: 32 - protein, 12 aa, Artificial Sequence
  Synthetic construct
RTNAADHPAAVT

SEQ ID NO: 33 - protein, 12 aa, Artificial Sequence
  Synthetic construct
SLNWVTIPGPKI

SEQ ID NO: 34 - protein, 12 aa, Artificial Sequence
  Synthetic construct
TDMQAPTKSYSN

SEQ ID NO: 35 - protein, 12 aa, Artificial Sequence
  Synthetic construct
TIMTKSPSLSCG

SEQ ID NO: 36 - protein, 12 aa, Artificial Sequence
  Synthetic construct
TPALDGLRQPLR

SEQ ID NO: 37 - protein, 12 aa, Artificial Sequence
  Synthetic construct
TYPASRLPLLAP

SEQ ID NO: 38 - protein, 12 aa, Artificial Sequence
  Synthetic construct
AKTHKHPAPSYS

SEQ ID NO: 39 - protein, 12 aa, Artificial Sequence
  Synthetic construct
TDPTPFSISPER

SEQ ID NO: 40 - protein, 12 aa, Artificial Sequence
  Synthetic construct
SQNWQDSTSYSN

SEQ ID NO: 41 - protein, 12 aa, Artificial Sequence
  Synthetic construct
WHDKPQNSSKST

SEQ ID NO: 42 - protein, 12 aa, Artificial Sequence
  Synthetic construct
LDVESYKGTSMP

SEQ ID NO: 43 - protein, 7 aa, Artificial Sequence
  Synthetic construct
NTPKENW

SEQ ID NO: 44 - protein, 7 aa, Artificial Sequence
  Synthetic construct
NTPASNR

SEQ ID NO: 45 - protein, 7 aa, Artificial Sequence
  Synthetic construct
PRGMLST

SEQ ID NO: 46 - protein, 7 aa, Artificial Sequence
  Synthetic construct
PPTYLST

SEQ ID NO: 47 - protein, 12 aa, Artificial Sequence
  Synthetic construct
TIPTHRQHDYRS

SEQ ID NO: 48 - protein, 7 aa, Artificial Sequence
  Synthetic construct
TPPTHRL

SEQ ID NO: 49 - protein, 7 aa, Artificial Sequence
  Synthetic construct
LPTMSTP

SEQ ID NO: 50 - protein, 7 aa, Artificial Sequence
  Synthetic construct
LGTNSTP

SEQ ID NO: 51 - protein, 12 aa, Artificial Sequence
  Synthetic construct
TPLTGSTNLLSS

SEQ ID NO: 52 - protein, 7 aa, Artificial Sequence
  Synthetic construct
TPLTKET

SEQ ID NO: 53 - protein, 7 aa, Artificial Sequence
  Synthetic construct
KQSHNPP

SEQ ID NO: 54 - protein, 7 aa, Artificial Sequence
  Synthetic construct
QQSHNPP

SEQ ID NO: 55 - protein, 7 aa, Artificial Sequence
  Synthetic construct
TQPHNPP

SEQ ID NO: 56 - protein, 12 aa, Artificial Sequence
  Synthetic construct
STNLLRTSTVHP

SEQ ID NO: 57 - protein, 12 aa, Artificial Sequence
  Synthetic construct
HTQPSYSSTNLF

SEQ ID NO: 58 - protein, 7 aa, Artificial Sequence
  Synthetic construct
SLLSSHA

SEQ ID NO: 59 - protein, 12 aa, Artificial Sequence
  Synthetic construct
QQSSISLSSHAV

SEQ ID NO: 60 - protein, 7 aa, Artificial Sequence
  Synthetic construct
NASPSSL

SEQ ID NO: 61 - protein, 7 aa, Artificial Sequence
  Synthetic construct
HSPSSLR

SEQ ID NO: 62 - protein, 7 aa, Artificial Sequence
  Synthetic construct
KXSHHTH

SEQ ID NO: 63 - protein, 7 aa, Artificial Sequence
  Synthetic construct
EXSHHTH

SEQ ID NO: 64 - protein, 12 aa, Artificial Sequence
  Synthetic construct
SHHTHYGQPGPV

SEQ ID NO: 65 - protein, 7 aa, Artificial Sequence
  Synthetic construct
LESTSLL

SEQ ID NO: 66 - protein, 7 aa, Artificial Sequence
  Synthetic construct
DLTLPFH

SEQ ID NO: 67 - protein, 8 aa, Artificial Sequence
  Synthetic construct
RTNAADHP

SEQ ID NO: 68 - protein, 12 aa, Artificial Sequence
  Synthetic construct
IPWWNIRAPLNA

SEQ ID NO: 69 - protein, 18 aa, Artificial Sequence
  Synthetic construct
EQISGSLVAAPWEGEGER

SEQ ID NO: 70 - protein, 29 aa, Artificial Sequence
  Synthetic construct
PKRGRHKRPKRHKGGGTPPELLHGAPRSC

SEQ ID NO: 71 - protein, 18 aa, Artificial Sequence
  Synthetic construct
LDTSFHQVPFHQKRKRKD

SEQ ID NO: 72 - protein, 18 aa, Artificial Sequence
  Synthetic construct
EQISGSLVAAPWKRKRKD

SEQ ID NO: 73 - protein, 18 aa, Artificial Sequence
  Synthetic construct
TPPELLHGDPRSKRKRKD

SEQ ID NO: 74 - protein, 13 aa, Artificial Sequence
  Synthetic construct
NTSQLSTEGEGED

SEQ ID NO: 75 - protein, 13 aa, Artificial Sequence
  Synthetic construct
TPPELLHGDPRSC

SEQ ID NO: 76 - protein, 20 aa, Artificial Sequence
  Synthetic construct
HINKTNPHQGNHHSEKTQRQ

SEQ ID NO: 77 - protein, 15 aa, Artificial Sequence
  Synthetic construct
HAHKNQKETHQRHAA

SEQ ID NO: 78 - protein, 15 aa, Artificial Sequence
  Synthetic construct
HEHKNQKETHQRHAA

SEQ ID NO: 79 - protein, 20 aa, Artificial Sequence
  Synthetic construct
HNHMQERYTEPQHSPSVNGL

SEQ ID NO: 80 - protein, 17 aa, Artificial Sequence
  Synthetic construct
THSTHNHGSPRHTNADA

SEQ ID NO: 81 - protein, 20 aa, Artificial Sequence
  Synthetic construct
GSCVDTHKADSCVANNGPAT

SEQ ID NO: 82 - protein, 20 aa, Artificial Sequence
  Synthetic construct
AQSQLPDKHSGLHERAPQRY

SEQ ID NO: 83 - protein, 20 aa, Artificial Sequence
  Synthetic construct
AQSQLPAKHSGLHERAPQRY

SEQ ID NO: 84 - protein, 20 aa, Artificial Sequence
  Synthetic construct
AQSQLPEKHSGLHERAPQRY

SEQ ID NO: 85 - protein, 20 aa, Artificial Sequence
  Synthetic construct
TDMMHNHSDNSPPHRRSPRN

SEQ ID NO: 86 - protein, 20 aa, Artificial Sequence
  Synthetic construct
TPPELAHTPHHLAQTRLTDR

SEQ ID NO: 87 - protein, 12 aa, Artificial Sequence
  Synthetic construct
RLLRLLRLLRLL

SEQ ID NO: 88 - protein, 12 aa, Artificial Sequence
  Synthetic construct
TPPELLHGEPRS

SEQ ID NO: 89 - protein, 12 aa, Artificial Sequence
  Synthetic construct
TPPELLHGAPRS

SEQ ID NO: 90 - protein, 12 aa, Artificial Sequence
  Synthetic construct
EQISGSLVAAPW

SEQ ID NO: 91 - protein, 12 aa, Artificial Sequence
  Synthetic construct
NEVPARNAPWLV

SEQ ID NO: 92 - protein, 13 aa, Artificial Sequence
  Synthetic construct
NSPGYQADSVAIG

SEQ ID NO: 93 - protein, 12 aa, Artificial Sequence
  Synthetic construct
AKPISQHLQRGS

SEQ ID NO: 94 - protein, 12 aa, Artificial Sequence
  Synthetic construct
LDTSFPPVPFHA

SEQ ID NO: 95 - protein, 12 aa, Artificial Sequence
  Synthetic construct
SLNWVTIPGPKI

SEQ ID NO: 96 - protein, 12 aa, Artificial Sequence
  Synthetic construct
TQDSAQKSPSPL

SEQ ID NO: 97 - protein, 12 aa, Artificial Sequence
  Synthetic construct
KELQTRNVVQRE

SEQ ID NO: 98 - protein, 12 aa, Artificial Sequence
  Synthetic construct
QRNSPPAMSRRD

SEQ ID NO: 99 - protein, 12 aa, Artificial Sequence
  Synthetic construct
TPTANQFTQSVP

SEQ ID NO: 100 - protein, 12 aa, Artificial Sequence
  Synthetic construct
AAGLSQKHERNR

SEQ ID NO: 101 - protein, 12 aa, Artificial Sequence
  Synthetic construct
ETVHQTPLSDRP

SEQ ID NO: 102 - protein, 12 aa, Artificial Sequence
  Synthetic construct
KNFPQQKEFPLS

SEQ ID NO: 103 - protein, 12 aa, Artificial Sequence
  Synthetic construct
LPALHIQRHPRM

SEQ ID NO: 104 - protein, 12 aa, Artificial Sequence
  Synthetic construct
QPSHSQSHNLRS

SEQ ID NO: 105 - protein, 12 aa, Artificial Sequence
  Synthetic construct
RGSQKSKPPRPP

SEQ ID NO: 106 - protein, 12 aa, Artificial Sequence
  Synthetic construct
THTQKTPLLYYH

SEQ ID NO: 107 - protein, 12 aa, Artificial Sequence
  Synthetic construct
TKGSSQAILKST

SEQ ID NO: 108 - protein, 7 aa, Artificial Sequence
  Synthetic construct
TAATTSP

SEQ ID NO: 109 - protein, 7 aa, Artificial Sequence
  Synthetic construct
LGIPQNL

SEQ ID NO: 110 - protein, 20 aa, Artificial Sequence
  Synthetic construct
THSTHNHGSPRHTNADAGNP

SEQ ID NO: 111 - protein, 20 aa, Artificial Sequence
  Synthetic construct
QQHKVHHQNPDRSTQDAHHS

SEQ ID NO: 112 - protein, 15 aa, Artificial Sequence
  Synthetic construct
HHGTHHNATKQKNHV

SEQ ID NO: 113 - protein, 15 aa, Artificial Sequence
  Synthetic construct
STLHKYKSQDPTPHH

SEQ ID NO: 114 - protein, 12 aa, Artificial Sequence
  Synthetic construct
SVSVGMKPSPRP

SEQ ID NO: 115 - protein, 12 aa, Artificial Sequence
  Synthetic construct
TPPTNVLMLATK

SEQ ID NO: 116 - protein, 12 aa, Artificial Sequence
  Synthetic construct
TPPELLHGDPRS

SEQ ID NO: 117 - protein, 7 aa, Artificial Sequence
  Synthetic construct
NTSQLST

SEQ ID NO: 118 - protein, 15 aa, Artificial Sequence
  Synthetic construct
STLHKYKSQDPTPHH

SEQ ID NO: 119 - protein, 12 aa, Artificial Sequence
  Synthetic construct
GMPAMHWIHPFA

SEQ ID NO: 120 - protein, 15 aa, Artificial Sequence
  Synthetic construct
HDHKNQKETHQRHAA

SEQ ID NO: 121 - protein, 20 aa, Artificial Sequence
  Synthetic construct
HNHMQERYTDPQHSPSVNGL

SEQ ID NO: 122 - protein, 20 aa, Artificial Sequence
  Synthetic construct
TAEIQSSKNPNPHPQRSWTN

SEQ ID NO: 123 - protein, 67 aa, Artificial Sequence
  Synthetic construct
PNTSQLSTGGGRTNAADHPKCGGGNTSQLSTGGGRTNAADHPKCGGGNTSQLSTGGGRTNAADHPKC

SEQ ID NO: 124 - protein, 55 aa, Artificial Sequence
  Synthetic construct
PRTNAADHPAAVTGGGCGGGRTNAADHPAAVTGGGCGGGRTNAADHPAAVTGGGC

SEQ ID NO: 125 - protein, 50 aa, Artificial Sequence
  Synthetic construct
PRTNAADHPAAVTGGGCGGGIPWWNIRAPLNAGGGCGGGDLTLPFHGGGC

SEQ ID NO: 126 - protein, 82 aa, Artificial Sequence
  Synthetic construct
PRTNAADHPGGGTPPELLHGDPRSKCGGGRTNAADHPGGGTPPELLHGDPRSKCGGGRTNAADHPGGGTPPELLHGDPRSKC

SEQ ID NO: 127 - protein, 82 aa, Artificial Sequence
  Synthetic construct
PTPPTNVLMLATKGGGRTNAADHPKCGGGTPPTNVLMLATKGGGRTNAADHPKCGGGTPPTNVLMLATKGGGRTNAADHPKC

SEQ ID NO: 128 - protein, 82 aa, Artificial Sequence
  Synthetic construct
PRTNAADHPGGGTPPTNVLMLATKKCGGGRTNAADHPGGGTPPTNVLMLATKKCGGGRTNAADHPGGGTPPTNVLMLATKKC

SEQ ID NO: 129 - protein, 60 aa, Artificial Sequence
  Synthetic construct
PGIPWWNIRAPLNAGAGIPWWNIRAPLNAGGSGPGSGGNTSQLSTGGGNTSQLSTGGPKK

SEQ ID NO: 130 - protein, 12 aa, Artificial Sequence
  Synthetic construct
KRGRHKRPKRHK

SEQ ID NO: 131 - protein, 7 aa, Artificial Sequence
  Synthetic construct
RLLRLLR

SEQ ID NO: 132 - protein, 12 aa, Artificial Sequence
  Synthetic construct
HKPRGGRKKALH

SEQ ID NO: 133 - protein, 18 aa, Artificial Sequence
  Synthetic construct
KPRPPHGKKHRPKHRPKK

SEQ ID NO: 134 - protein, 18 aa, Artificial Sequence
  Synthetic construct
RGRPKKGHGKRPGHRARK

SEQ ID NO: 135 - protein, 12 aa, Artificial Sequence
  Synthetic construct
TPFHSPENAPGS

SEQ ID NO: 136 - protein, 13 aa, Artificial Sequence
  Synthetic construct
TPFHSPENAPGSK

SEQ ID NO: 137 - protein, 16 aa, Artificial Sequence
  Synthetic construct
TPFHSPENAPGSGGGS

SEQ ID NO: 138 - protein, 17 aa, Artificial Sequence
  Synthetic construct
TPFHSPENAPGSGGGSS

SEQ ID NO: 139 - protein, 15 aa, Artificial Sequence
  Synthetic construct
TPFHSPENAPGSGGG

SEQ ID NO: 140 - protein, 7 aa, Artificial Sequence
  Synthetic construct
FTQSLPR

SEQ ID NO: 141 - protein, 12 aa, Artificial Sequence
  Synthetic construct
KQATFPPNPTAY

SEQ ID NO: 142 - protein, 12 aa, Artificial Sequence
  Synthetic construct
HGHMVSTSQLSI

SEQ ID NO: 143 - protein, 7 aa, Artificial Sequence
  Synthetic construct
LSPSRMK

SEQ ID NO: 144 - protein, 7 aa, Artificial Sequence
  Synthetic construct
LPIPRMK

SEQ ID NO: 145 - protein, 7 aa, Artificial Sequence
  Synthetic construct
HQRPYLT

SEQ ID NO: 146 - protein, 7 aa, Artificial Sequence
  Synthetic construct
FPPLLRL

SEQ ID NO: 147 - protein, 7 aa, Artificial Sequence
  Synthetic construct
QATFMYN

SEQ ID NO: 148 - protein, 11 aa, Artificial Sequence
  Synthetic construct
VLTSQLPNHSM

SEQ ID NO: 149 - protein, 7 aa, Artificial Sequence
  Synthetic construct
HSTAYLT

SEQ ID NO: 150 - protein, 12 aa, Artificial Sequence
  Synthetic construct
APQQRPMKTFNT

SEQ ID NO: 151 - protein, 12 aa, Artificial Sequence
  Synthetic construct
APQQRPMKTVQY

SEQ ID NO: 152 - protein, 7 aa, Artificial Sequence
  Synthetic construct
PPWLDLL

SEQ ID NO: 153 - protein, 7 aa, Artificial Sequence
  Synthetic construct
PPWTFPL

SEQ ID NO: 154 - protein, 7 aa, Artificial Sequence
  Synthetic construct
SVTHLTS

SEQ ID NO: 155 - protein, 7 aa, Artificial Sequence
  Synthetic construct
VITRLTS

SEQ ID NO: 156 - protein, 12 aa, Artificial Sequence
  Synthetic construct
DLKPPLLALSKV

SEQ ID NO: 157 - protein, 12 aa, Artificial Sequence
  Synthetic construct
SHPSGALQEGTF

SEQ ID NO: 158 - protein, 12 aa, Artificial Sequence
  Synthetic construct
FPLTSKPSGACT

SEQ ID NO: 159 - protein, 12 aa, Artificial Sequence
  Synthetic construct
DLKPPLLALSKV

SEQ ID NO: 160 - protein, 7 aa, Artificial Sequence
  Synthetic construct
PLLALHS

SEQ ID NO: 161 - protein, 7 aa, Artificial Sequence
  Synthetic construct
VPISTQI

SEQ ID NO: 162 - protein, 12 aa, Artificial Sequence
  Synthetic construct
YAKQHYPISTFK

SEQ ID NO: 163 - protein, 7 aa, Artificial Sequence
  Synthetic construct
HSTAYLT

SEQ ID NO: 164 - protein, 12 aa, Artificial Sequence
  Synthetic construct
STAYLVAMSAAP

SEQ ID NO: 165 - protein, 12 aa, Artificial Sequence
  Synthetic construct
SVSVGMKPSPRP

SEQ ID NO: 166 - protein, 12 aa, Artificial Sequence
  Synthetic construct
TMGFTAPRFPHY

SEQ ID NO: 167 - protein, 12 aa, Artificial Sequence
  Synthetic construct
NLQHSVGTSPVW

SEQ ID NO: 168 - protein, 15 aa, Artificial Sequence
  Synthetic construct
QLSYHAYPQANHHAP

SEQ ID NO: 169 - protein, 12 aa, Artificial Sequence
  Synthetic construct
NQAASITKRVPY

SEQ ID NO: 170 - protein, 14 aa, Artificial Sequence
  Synthetic construct
SGCHLVYDNGFCDH

SEQ ID NO: 171 - protein, 14 aa, Artificial Sequence
  Synthetic construct
ASCPSASHADPCAH

SEQ ID NO: 172 - protein, 14 aa, Artificial Sequence
  Synthetic construct
NLCDSARDSPRCKV

SEQ ID NO: 173 - protein, 12 aa, Artificial Sequence
  Synthetic construct
NHSNWKTAADFL

SEQ ID NO: 174 - protein, 12 aa, Artificial Sequence
  Synthetic construct
GSSTVGRPLSYE

SEQ ID NO: 175 - protein, 12 aa, Artificial Sequence
  Synthetic construct
SDTISRLHVSMT

SEQ ID NO: 176 - protein, 12 aa, Artificial Sequence
  Synthetic construct
SPLTVPYERKLL

SEQ ID NO: 177 - protein, 12 aa, Artificial Sequence
  Synthetic construct
SPYPSWSTPAGR

SEQ ID NO: 178 - protein, 12 aa, Artificial Sequence
  Synthetic construct
VQPITNTRYEGG

SEQ ID NO: 179 - protein, 12 aa, Artificial Sequence
  Synthetic construct
WPMHPEKGSRWS

SEQ ID NO: 180 - protein, 14 aa, Artificial Sequence
  Synthetic construct
DACSGNGHPNNCDR

SEQ ID NO: 181 - protein, 14 aa, Artificial Sequence
  Synthetic construct
DHCLGRQLQPVCYP

SEQ ID NO: 182 - protein, 14 aa, Artificial Sequence
  Synthetic construct
DWCDTIIPGRTCHG

SEQ ID NO: 183 - protein, 12 aa, Artificial Sequence
  Synthetic construct
ALPRIANTWSPS

SEQ ID NO: 184 - protein, 12 aa, Artificial Sequence
  Synthetic construct
YPSFSPTYRPAF

SEQ ID NO: 185 - protein, 19 aa, Artificial Sequence
  Synthetic or a naturally-occurring histidine affinity tag; metal binding peptide
KDHLIHNVHKEFHAHAHNK

SEQ ID NO: 186 - protein, 21 aa, Artificial Sequence
  Synthetic hair-binding peptide
HINKTNPHQGNHHSEKTQRQK

SEQ ID NO: 187 - protein, 21 aa, Artificial Sequence
  Synthetic hair-binding peptide
AQSQLPDKHSGLHERAPQRYK

SEQ ID NO: 188 - protein, 21 aa, Artificial Sequence
  Synthetic hair-binding peptide
TPPELAHTPHHLAQTRLTDRK

SEQ ID NO: 189 - protein, 21 aa, Artificial Sequence
  Synthetic hair-binding peptide
THSTHNHGSPRHTNADAGNPK

SEQ ID NO: 190 - protein, 20 aa, Artificial Sequence
  synthetic construct
DRSKLYQSLEHRQPPGAHAQ

SEQ ID NO: 191 - protein, 15 aa, Artificial Sequence
  synthetic construct
AHDAKHEHRKQNHTQ

SEQ ID NO: 192 - protein, 12 aa, Artificial Sequence
  synthetic construct
QTHVSQPFLFHD

SEQ ID NO: 193 - protein, 12 aa, Artificial Sequence
  Synthetic pigment binding peptide
WAPEKDHMQLMK

SEQ ID NO: 194 - protein, 20 aa, Artificial Sequence
  Synthetic cellulose acetate-binding peptide
NGNNHTDIPNRSSYTGGSFA

SEQ ID NO: 195 - protein, 15 aa, Artificial Sequence
  Synthetic cellulose acetate-binding peptide
SDETGPQIPHRRPTW

SEQ ID NO: 196 - protein, 94 aa, Artificial Sequence
  synthetic construct
MASGSDPSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 197 - protein, 94 aa, Artificial Sequence
  synthetic construct
MASGSDPSDRSKLYQSLEHRQPPGAHAQGPEPEPEPEPIPEPPKEAPVVIEKPKPKPKPKPKPPAAHDAKHEHRKQNHTQGSGGGGSPHHHHHH

SEQ ID NO: 198 - protein, 55 aa, Artificial Sequence
  synthetic construct
GSDPGHDHKNQKETHQRHAAGGGHDHKNQKETHQRHAAGGGGSGGGGSGHHHHHH

SEQ ID NO: 199 - protein, 49 aa, Artificial Sequence
  synthetic construct
GSDPGQTHVSQPFLFHDGGGQTHVSQPFLFHDGGGGSGGGGSGHHHHHH

SEQ ID NO: 200 - protein, 34 aa, Artificial Sequence
  synthetic construct
GSDPGTPPELLHGAPRSGGGGSGGGGSGHHHHHH

SEQ ID NO: 201 - protein, 121 aa, Artificial Sequence
  synthetic construct
MASGSDPSAQSQLPDKHSGLHERAPQRYGPEPEPEPEPIPEPPKEAPVVIEKPKPKPKPKPKPPAHDHKNQKETHQRHAAGSGGGGSPWAPEKDHMQLMKGGAGGAGWAPEKDHMQLMKGK

SEQ ID NO: 202 - protein, 149 aa, Artificial Sequence
  synthetic construct
MASGSDPSAQSQLPDKHSGLHERAPQRYGPEPEPEPEPIPEPPKEAPVVIEKPKPKPKPKPKPPAHDHKNQKETHQRHAAGPGGGSGGGSGGGSGGGSPANGNNHTDIPNRSSYTGGSFAGGAGGAGNGNNHTDIPNRSSYTGGSFAGK

SEQ ID NO: 203 - protein, 139 aa, Artificial Sequence
  synthetic construct
MASGSDPSAQSQLPDKHSGLHERAPQRYGPEPEPEPEPIPEPPKEAPVVIEKPKPKPKPKPKPPAHDHKNQKETHQRHAAGPGGGSGGGSGGGSGGGSPASDETGPQIPHRRPTWGGAGGAGSDETGPQIPHRRPTWGK

SEQ ID NO: 204 - protein, 9 aa, Artificial Sequence
  synthetic construct
GGSGPGSGG

SEQ ID NO: 205 - protein, 9 aa, Artificial Sequence
  synthetic construct
GGSGPGSGG

SEQ ID NO: 206 - protein, 13 aa, Artificial Sequence
  Synthetic streptavidin binding peptide
MLSENWLTNHPQN

SEQ ID NO: 207 - protein, 123 aa, Artificial Sequence
  synthetic construct
MASGSDPSAQSQLPDKHSGLHERAPQRYGPEPEPEPEPIPEPPKEAPVVIEKPKPKPKPKPKPPAHDHKNQKETHQRHAAGSGGGGSPMLSENWLTNHPQNGGAGGAGMLSENWLTNHPQNGK

SEQ ID NO: 208 - protein, 35 aa, Artificial Sequence
  Synthetic peptide linker
GEPEPEPEPIPEPPKEAPVVIEKPKPKPKPKPKPA

SEQ ID NO: 209 - protein, 16 aa, Artificial Sequence
  synthetic construct
GSGGGGSPGSGGGGSP

SEQ ID NO: 210 - protein, 135 aa, Artificial Sequence
  synthetic construct
MASGSDPSAQSQLPDKHSGLHERAPQRYGPEPEPEPEPIPEPPKEAPVVIEKPKPKPKPKPKPPAHDHKNQKETHQRHAAGSGGGGSPWAPEKDHMQLMKGKGKGKGKGKGKGKGKGKGKGWAPEKDHMQLMKGK

SEQ ID NO: 211 - protein, 104 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPKQPNKQPNKQPNKQPNKQPN

SEQ ID NO: 212 - protein, 120 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPKQPNKQPNKQPNKQPNKQPNKQPNKQPNKQPNKQPN

SEQ ID NO: 213 - protein, 136 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPKQPNKQPNKQPNKQPNKQPNKQPNKQPNKQPNKQPNKQPNKQPNKQPNKQPN

SEQ ID NO: 214 - protein, 129 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPKQGPQKQGPQKQGPQKQGPQKQGPQKQGPQKQGPQKQGPQKQGPQ

SEQ ID NO: 215 - protein, 138 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPKQGPQGKQGPQGKQGPQGKQGPQGKQGPQGKQGPQGKQGPQGKQGPQGKQGPQG

SEQ ID NO: 216 - protein, 147 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPKGQGPQGKGQGPQGKGQGPQGKGQGPQGKGQGPQGKGQGPQGKGQGPQGKGQGPQGKGQGPQG

SEQ ID NO: 217 - protein, 124 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPKQPNHQPQKQPNHQPQKQPNHQPQKQPNHQPQKQPNHQPQ

SEQ ID NO: 218 - protein, 124 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPHQPNHQPQHQPNHQPQHQPNHQPQHQPNHQPQHQPNHQPQ

SEQ ID NO: 219 - protein, 124 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPHSPNHSPSHSPNHSPSHSPNHSPSHSPNHSPSHSPNHSPS

SEQ ID NO: 220 - protein, 108 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPHTSPQHHTSPHPHKNPQHTSPTPN

SEQ ID NO: 221 - protein, 108 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPHTKQNHTSPTPHHTNSPQHTSPHP

SEQ ID NO: 222 - protein, 108 aa, Artificial Sequence
  synthetic construct
AQSQLPEKHSGLHERAPQRYEPEPEPEPIPEPPKEAPVVIEKPKPKPKPKPKPHAHKNQKETHQRHAAGSGGGGSPGSGGGGSPHNNRQHTSPHPQHTKQNHTSPTPH

SEQ ID NO: 223 - protein, 20 aa, Artificial Sequence
  synthetic construct
KQPNKQPNKQPNKQPNKQPN

SEQ ID NO: 224 - protein, 36 aa, Artificial Sequence
  synthetic construct
KQPNKQPNKQPNKQPNKQPNKQPNKQPNKQPNKQPN

SEQ ID NO: 225 - protein, 52 aa, Artificial Sequence
  synthetic construct
KQPNKQPNKQPNKQPNKQPNKQPNKQPNKQPNKQPNKQPNKQPNKQPNKQPN

SEQ ID NO: 226 - protein, 45 aa, Artificial Sequence
  synthetic construct
KQGPQKQGPQKQGPQKQGPQKQGPQKQGPQKQGPQKQGPQKQGPQ

SEQ ID NO: 227 - protein, 54 aa, Artificial Sequence
  synthetic construct
KQGPQGKQGPQGKQGPQGKQGPQGKQGPQGKQGPQGKQGPQGKQGPQGKQGPQG

SEQ ID NO: 228 - protein, 63 aa, Artificial Sequence
  synthetic construct
KGQGPQGKGQGPQGKGQGPQGKGQGPQGKGQGPQGKGQGPQGKGQGPQGKGQGPQGKGQGPQG

SEQ ID NO: 229 - protein, 40 aa, Artificial Sequence
  synthetic construct
KQPNHQPQKQPNHQPQKQPNHQPQKQPNHQPQKQPNHQPQ

SEQ ID NO: 230 - protein, 40 aa, Artificial Sequence
  synthetic construct
HQPNHQPQHQPNHQPQHQPNHQPQHQPNHQPQHQPNHQPQ

SEQ ID NO: 231 - protein, 40 aa, Artificial Sequence
  synthetic construct
HSPNHSPSHSPNHSPSHSPNHSPSHSPNHSPSHSPNHSPS

SEQ ID NO: 232 - protein, 24 aa, Artificial Sequence
  synthetic construct
HTSPQHHTSPHPHKNPQHTSPTPN

SEQ ID NO: 233 - protein, 24 aa, Artificial Sequence
  synthetic construct
HTKQNHTSPTPHHTNSPQHTSPHP

SEQ ID NO: 234 - protein, 24 aa, Artificial Sequence
  synthetic construct
HNNRQHTSPHPQHTKQNHTSPTPH

SEQ ID NO: 235 - protein, 6 aa, Artificial Sequence
  Synthetic polyhistidine tag
HHHHHH